(19) United States
(12) Patent Application Publication
(10) Pub. No.: US 2015/0119402 A1
(43) Pub. Date: Apr. 30, 2015

(54) ALPHA 7 NICOTINIC ACETYLCHOLINE ALLOSTERIC MODULATORS, THEIR DERIVATIVES AND USES THEREOF

(71) Applicant: THE REGENTS OF THE UNIVERSITY OF CALIFORNIA, Oakland, CA (US)

(72) Inventor: Derk Hogenkamp, Carlsbad, CA (US)

(21) Appl. No.: 14/399,479

(22) PCT Filed: May 8, 2013
(86) PCT No.: PCT/US2013/040153

§ 371 Date: Nov. 6, 2014

Related U.S. Application Data
(60) Provisional application No. 61/644,414, filed on May 8, 2012.
(60) Provisional application No. 61/646,122, filed on May 11, 2012.

(51) Int. Cl. C07D 401/06; C07D 403/06

(52) U.S. Cl. CPC C07D 401/06; C07D 403/06
USPC 514/252.06; 546/278.1; 514/339; 546/275.7; 514/338; 544/405; 514/255.05; 544/238

(57) ABSTRACT

The present application is related to compounds represented by Formula I, which are novel positive allosteric modulators of al nAChRs. The application also discloses the treatment of disorders that are responsive to enhancement of acetylcholine action on al nAChRs in a mammal by administering an effective amount of a compound of Formula I.

### RELATED APPLICATIONS

The present application claims the benefit of priority to U.S. Ser. No. 61/644,414, and filed on May 8, 2012; and U.S. Ser. No. 61/646,122, and filed on May 11, 2012 and are incorporated by reference in their entirety.

### GOVERNMENT LICENSE RIGHTS

This invention was made with U.S. government support under grant R21-MH082241 awarded by National Institute of Mental Health. The U.S. government has certain rights in the invention.

### BACKGROUND

The disclosure of the present application is in the field of medicinal chemistry. In particular, this application discloses a class of novel compounds that allosterically modulate the α7 nicotinic acetylcholine receptor (α7 nAChR) and may be used to treat disorders amenable to modulation of the α7 nAChR.

α7 nAChRs belong to the ligand-gated ion channel superfamily of Cys-loop receptors. The Cys-loop superfamily includes muscle and neuronal nAChRs, 5-hydroxytryptamine type 3 (5HT₃), γ-aminobutyric acid_{A} (GABA_{A}), GABA_{C} and glycine receptors. α7 nAChRs are ion channels that recognize acetylcholine and choline as the endogenous orthosteric ligand and also bind nicotine at the orthosteric site. α7 nAChRs contain 5 orthosteric receptor sites per receptor. Agonist binding to the orthosteric site effects functional states of the receptor depending on the concentration and kinetics of agonist application. Four functional states have been described for α7 nAChRs: one open and three closed states (resting, fast-onset desensitized, slow-onset desensitized). Unlike agonists, allosteric modulators of α7 nAChRs do not bind to the orthosteric site, and cannot affect the functional state of the ion channel by themselves. An allosteric modulator of α7 nAChRs requires the presence of an agonist to activate the channel, and in-turn potentiates the action of the agonist. In the brain, activation of neuronal α7 nAChRs mediates fast synaptic transmission and controls synaptic transmission by the major inhibitory and excitatory neurotransmitters, GABA and glutamate.

α7 nAChRs mediate the predominant nicotinic current in hippocampal neurons. The α7 nAChR was initially identified from a chick brain library as an α-bungarotoxin binding protein that exhibits 40% sequence homology to other nAChRs. α7 nAChRs share similar features of other neuronal and muscle nAChRs such as a pentameric Cys-loop receptor structure and M2 segment of each subunit lining of the channel pore, however the α7 nAChRs exhibits a homopentameric structure when reconstituted in Xenopus oocytes, a characteristic shared only with the a8 and a9 nAChRs. Heterologously expressed homomeric α7 nAChRs in Xenopus oocytes are inactivated by α-bungarotoxin with high affinity, whereas other nAChRs are not. α7 nAChRs have also been pharmacologically identified by distinct types of whole cell currents elicited by nicotinic agonists in hippocampal neurons. When exposed to various nicotinic agonists, whole cell recordings from cultured hippocampal neurons show, in general, type IA currents that have a very brief open time, high conductance, very high Ca⁺⁺ permeability, decay rapidly, and are sensitive to blockade by methyllycaconitine (MLA) and α-bungarotoxin. The properties of these nicotinic currents in hippocampal neurons correspond to the currents mediated by α7 nAChRs expressed in oocytes.

### SUMMARY OF THE INVENTION

Briefly, this invention is generally directed to allosteric modulators of the α7 nAChR, as well as to methods for their preparation and use, and to pharmaceutical compositions containing the same. More specifically, the allosteric α7 nAChR modulators of this invention are compounds represented by the general structure:

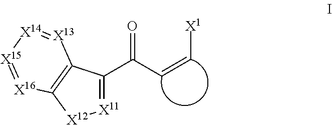

including pharmaceutically acceptable salts, solvates, and prodrugs thereof, wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and X¹, X², X³, X⁴, X⁵, X⁶, X⁷, X⁸, X⁹, X¹⁰, X¹¹, X¹², X¹³, X¹⁴, X¹⁵, and X¹⁶ are as defined below.

Further, the present invention is directed to ²H, ³H, ¹¹C, ¹⁸F, ³⁵S, ³⁶Cl, ¹⁴C and ¹²⁵I labeled compounds of Formulae I-VII and their use as stably isotopically labeled analogs or as radioligands for their binding site on the α7 nAChR complex.

This invention also is directed to methods of treating disorders responsive to enhancement of acetylcholine action on α7 nAChRs in a mammal by administering an effective amount of a compound of Formulae I-VII as described herein. Compounds of the present invention may be used to treat a variety of disorders, including of the central nervous system (CNS) and the peripheral nervous system (PNS). Such disorders of the CNS and the PNS include neurodegenerative diseases, senile dementias, schizophrenia, Alzheimer's disease, learning, cognition and attention deficits, memory loss, Lewy Body dementia, attention-deficit disorder, attention deficit hyperactivity disorder, anxiety, mania, manic depression, Parkinson's disease, Huntington's disease, depression, amyotrophic lateral sclerosis, brain inflammation, cognitive deficit due to traumatic brain injury, and Tourette's syndrome. Compounds of the invention are also useful in the treatment (therapeutic or prophylactic), prevention or delay of progression of dyskinesia associated with dopamine agonist therapy in Parkinson's disease. In addition, compounds of the present invention may be used to treat immune system disorders, such as, but not limited to, type I diabetes, multiple sclerosis, and rheumatoid arthritis. In addition, compounds of the present invention may be used to treat pain, inflammation, septic shock, ulcerative colitis, irritable bowel syndrome and Crohn's disease. Compounds of the invention are useful in tobacco cessation treatment (Brunzell et al. Neuropsychopharm. 2011, 1-10), in the treatment of diabetes (Marrero et al. JPET, 2009, 332, 173) and in treating jetlag. In addition, compounds of the invention are also of use in treating immune system disorders, Fragile X, autism spectrum disorder, Angelman's syndrome, Rett syndrome, Prader Willi syndrome and Down's syndrome.

The present invention also is directed to pharmaceutical formulations which include a compound of the present invention. Such formulations contain a therapeutically effective amount of a compound of Formulae I-VII, pharmaceuticlly acceptable salts, solvates, and prodrugs thereof, and one or more pharmaceutically acceptable carriers or diluents.

Additional embodiments and advantages of the invention will be set forth in part in the description that follows, and in part will be apparent from the description, or may be learned by practice of the invention. The embodiments and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### DETAILED DESCRIPTION

In one aspect, the present invention is directed to a compound of Formula I:

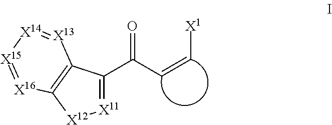

or pharmaceutically acceptable salts, solvates, and prodrugs thereof, wherein:

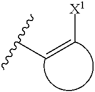

is a heteroaryl group selected from:

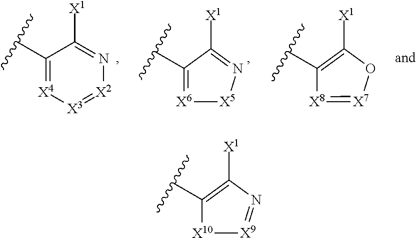

wherein

X¹ is O—R¹ or NH—R¹;

X² is N or C—R²;

X³ is N or C—R³;

X⁴ is N or C—R⁴;

X⁵ is O, S or N—R⁵;

X⁶ is N or N—R⁶;

X⁷ is Nor C—R⁷;

X⁸ is N or C—R⁸;

X⁹ is N or C—R⁹;

X¹⁰ is O, S or N—R¹⁰;

X¹¹ is N or C—R¹¹;

X¹² is O, S or N—R¹²;

X¹³ is N or C—R¹³;

X¹⁴ is N or C—R¹⁴;

X¹⁵ is N or C—R¹⁵;

X¹⁶ is N or C—R¹⁶;

R¹ is, C₁₋₈ alkyl, C₂₋₈ alkenyl, C₂₋₈ alkynyl, and C₁₋₈ haloalkyl, each optionally substituted; or

R¹ is aryl, heteroaryl, arylalkyl, heteroarylalkyl, C₃₋₈ cycloalkyl, cycloalkenyl, heterocycloalkyl and heterocycloalkenyl, each optionally substituted; and

R², R³, R⁴, R⁶, R⁷, R⁸, R⁹, R¹⁰, R¹³, R¹⁴, R¹⁵ and R¹⁶ are each independently selected from the group consisting of hydrogen, halogen, nitro, cyano, hydroxyl, amino, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₈ alkamino, C₁₋₈ haloalkamino, dialkylamino, alkenylamino, alkynylamino, arylamino, heteroarylamino, C₃₋₈ cycloalkamino, cycloalkenylamino, heterocycloalkylamino, heterocycloalkenylamino, C₁₋₈ alkthio, C₁₋₈ haloalkthio, alkenylthio, alkynylthio, arylthio, heteroarylthio, C₃₋₈ cycloalkthio, cycloalkenylthio, heterocycloalkylthio, heterocycloalkenylthio, —C(═O)R¹⁷, —N(R¹⁸)C(═O)R¹⁹, —OC(═O)R¹⁹, —N(R¹⁸)S(═O)₂R¹⁹, —S(═O)₂R¹⁷, and —S(═O)R¹⁷, each optionally substituted; and

R⁵, R¹⁰ and R¹² are independently selected from the group consisting of hydrogen, C₁₋₈ alkyl, C₃₋₈ cycloalkyl, C₃₋₈ alkenyl, C₃₋₈ alkynyl, C₁₋₈ haloalkyl, aryl, and heteroaryl; and

R² and R³, or R³ and R⁴, or R⁵ and R⁶, or R⁷ and R⁸, or R⁹ and R¹⁰, or R¹³ and R¹⁴, or R¹⁴ and R¹⁵ or R¹⁵ and R¹⁶ are taken together with the carbon atoms to which they are attached to form an unsubstituted or substituted fused 5 or 6-membered unsaturated or partially unsaturated ring optionally interrupted by one —O—, —NR²⁰—, —S—, —SO— or —SO₂—; and

each R¹⁷ is independently selected from the group consisting of hydroxyl, amino, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₆ alkylamino, C₁₋₈ haloalkylamino, dialkylamino, alkenylamino, alkynylamino, arylamino, heteroarylamino, C₃₋₈ cycloalkylamino, cycloalkenylamino, heterocycloalkylamino, and heterocycloalkenylamino, each optionally substituted; and

each R¹⁸ is independently selected from the group consisting of hydrogen, hydroxyl, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, and heterocycloalkenyloxy, each optionally substituted; and

each R¹⁹ is independently selected from the group consisting of amino, C₁₋₈ alkyl, C₂₋₆ alkenyl, C₂₋₆ alkynyl, C₁₋₆ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₈ alkylamino, C₁₋₈ haloalkylamino, dialkylamino, alkenylamino, alkynylamino, arylamino, heteroarylamino, C₃₋₈ cycloalkylamino, cycloalkenylamino, heterocycloalkylamino, and heterocycloalkenylamino, each optionally substituted; and

R²⁰ is selected from the group consisting of hydrogen, C₁₋₈ alkyl, C₃₋₈ cycloalkyl, C₃₋₈ alkenyl, C₃₋₈ alkynyl, C₁₋₈ haloalkyl, aryl, and heteroaryl.

### DEFINITIONS

Unless specifically noted otherwise herein, the definitions of the terms used are standard definitions used in the art of organic synthesis and pharmaceutical sciences.

The term “halogen” as used herein refers to a halogen radical selected from fluoro, chloro, bromo and iodo.

The term “cyano” refers to —C═N.

The term “nitro” refers to —NO₂.

The term “hydroxyl” refers to OH.

The term “alkyl” refers to a saturated aliphatic hydrocarbon radical. “Alkyl” refers to both branched and unbranched alkyl groups. One or more of the carbons may be oxidized to C(═O). Examples of “alkyl” include alkyl groups that are straight chain alkyl groups containing from one to ten carbon atoms and branched alkyl groups containing from three to ten carbon atoms. “Alkyl” includes but is not limited to straight chain alkyl groups containing from one to six carbon atoms and branched alkyl groups containing from three to six carbon atoms. This term is exemplified by groups such as methyl, ethyl, n-propyl, 1-methylethyl (isopropyl), 1,1-dimethylethyl (tert-butyl), and the like. It may be abbreviated “Alk”. It should be understood that any combination term using an “alk” or “alkyl” prefix refers to analogs according to the above definition of “alkyl” including the number of carbon atoms. For example, terms such as “alkoxy”, “alkylthio”, “alkylamino” refer to alkyl groups linked to a second group via an oxygen, sulfur, or nitrogen atom, respectively.

The term “haloalkyl” refers to an alkyl group in which one or more hydrogen atoms are replaced with halogen atoms. One or more of the carbons may be oxidized to C(═O). This term includes but is not limited to groups such as trifluoromethyl. In one embodiment the haloalkyl groups are alkyl groups substituted with one or more fluoro or chloro. The term “haloalkoxy” refers to haloalkyl groups linked to a second group via an oxygen atom.

The term “alkenyl” refers to a mono or polyunsaturated aliphatic hydrocarbon radical. The mono or polyunsaturated aliphatic hydrocarbon radical contains at least one carbon-carbon double bond. “Alkenyl” refers to both branched and unbranched alkenyl groups, each optionally partially or fully halogenated. One or more of the carbons may be oxidized to C(═O). Examples of “alkenyl” include alkenyl groups that are straight chain alkenyl groups containing from two to ten carbon atoms and branched alkenyl groups containing from three to ten carbon atoms. Other examples include alkenyl groups which are straight chain alkenyl groups containing from two to six carbon atoms and branched alkenyl groups containing from three to six carbon atoms. Alkenyl groups include but are not limited to ethenyl, propenyl, n-butenyl, isobutenyl, 3-methylbut-2-enyl, n-pentenyl, heptenyl, octenyl, decenyl, and the like. It should be understood that any combination term using an “alkenyl” prefix refers to analogs according to the above definition of “alkenyl” including the number of carbon atoms. For example, terms such as “alkenyloxy”, “alkenylthio”, “alkenylamino” refer to alkenyl groups linked to a second group via an oxygen, sulfur, or nitrogen atom, respectively.

The term “alkynyl” refers to a mono or polyunsaturated aliphatic hydrocarbon radical. The mono or polyunsaturated aliphatic hydrocarbon radical contains at least one carbon-carbon triple bond. “Alkynyl” refers to both branched and unbranched alkynyl groups, each optionally partially or fully halogenated. One or more of the carbons may be oxidized to C(═O). Examples of “alkynyl” include alkynyl groups that are straight chain alkynyl groups containing from two to ten carbon atoms and branched alkynyl groups containing from four to ten carbon atoms. Other examples include alkynyl groups that are straight chain alkynyl groups containing from two to six carbon atoms and branched alkynyl groups containing from four to six carbon atoms. This term is exemplified by groups such as ethynyl, propynyl, octynyl, and the like. It should be understood that any combination term using an “alkynyl” prefix refers to analogs according to the above definition of “alkynyl” including the number of carbon atoms. For example, terms such as “alkynyloxy”, “alkynylthio”, “alkynylamino” refer to alkynyl groups linked to a second group via an oxygen, sulfur, or nitrogen atom, respectively.

The term “cycloalkyl” refers to the mono- or polycyclic analogs of an alkyl group, as defined above. One or more of the carbons may be oxidized to C(═O). Unless otherwise specified, the cycloalkyl ring may be attached at any carbon atom that results in a stable structure and, if substituted, may be substituted at any suitable carbon atom which results in a stable structure. Examples of cycloalkyl groups are saturated cycloalkyl groups containing from three to ten carbon atoms. Other examples include cycloalkyl groups containing three to eight carbon atoms or three to six carbon atoms. Exemplary cycloalkyl groups include but are not limited to cyclopropyl, cyclobutyl, cyclopentyl, cyclohexyl, cyclooctyl, cyclononyl, cyclodecyl, norbornyl, adamantyl, and the like. It should be understood that any combination term using “cycloalkyl” refers to analogs according to the above definition of “cycloalkyl” including the number of carbon atoms. Terms such as “cycloalkyloxy”, “cycloalkylthio”, “cycloalkylamino” refer to a cycloalkyl groups linked to a second group via an oxygen, sulfur, or nitrogen atom, respectively.

The term “cycloalkenyl” refers to the mono- or polycyclic analogs of an alkenyl group, as defined above. One or more of the carbons may be oxidized to C(═O). Unless otherwise specified, the cycloalkenyl ring may be attached at any carbon atom that results in a stable structure and, if substituted, may be substituted at any suitable carbon atom which results in a stable structure. Examples of cycloalkenyl groups are cycloalkenyl groups containing from four to ten carbon atoms. Other examples include cycloalkenyl groups containing four to eight carbon atoms or four to six carbon atoms. Exemplary cycloalkenyl groups include but are not limited to cyclobutenyl, cyclopentenyl, cyclohexenyl, norbornenyl, and the like. It should be understood that any combination term using “cycloalkenyl” refers to analogs according to the above definition of “cycloalkenyl” including the number of carbon atoms. Terms such as “cycloalkenyloxy”, “cycloalkenylthio”, “cycloalkenylamino” refer to a cycloalkenyl groups linked to a second group via an oxygen, sulfur, or nitrogen atom, respectively.

The term “heterocycloalkyl” refers to the mono- or polycyclic structures of “cycloalkyl” where one or more of the carbon atoms are replaced by one or more atoms independently selected from nitrogen, oxygen, or sulfur atoms. Any nitrogen atom maybe optionally oxidized or quaternized, and any sulfur atom maybe optionally oxidized. Generally, the heteroatoms may be selected from the group consisting of N, S, S═O, S(═O)₂, and O. One or more of the carbons may be oxidized to C(═O). Unless otherwise specified, the heterocycloalkyl ring may be attached at any carbon atom or heteroatom that results in a stable structure and, if substituted, may be substituted at any suitable carbon atom or heteroatom which results in a stable structure. Examples of heterocycloalkyl groups are saturated heterocycloalkyl groups containing from two to nine carbon atoms and one to four heteroatoms. Generally, 5-7 membered heterocycloalkyl groups contain 3-6 carbon atoms and 1-2 heteroatoms independently selected from the group consisting of N, S, S═O, S(═O)₂, and O. Examples of heterocycloalkyl groups include but are not limited to morpholino, pyrazino, tetrahydrofurano, and the like. “Carbon-attached heterocycloalkyl” refers to a heterocycloalkyl group which is bound via a constituent carbon atom. A heterocycloalkyl that is fused with a phenyl can include, but is not limited to the following:

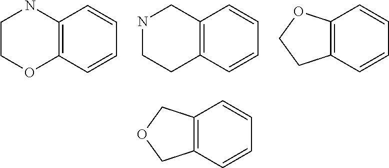

A heterocycloalkyl that is fused with a 5-6 membered heteroaryl can include, but is not limited to the following:

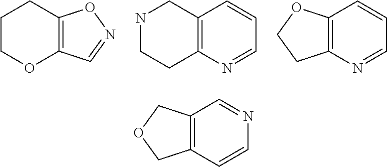

Terms such as “heterocycloalkyloxy”, “heterocycloalkylthio”, “heterocycloalkylamino” refer to heterocycloalkyl groups linked to a second group via an oxygen, sulfur, or nitrogen atom, respectively.

The term “heterocycloalkenyl” refers to the mono- or polycyclic structures of “cycloalkenyl” where one or more of the carbon atoms are replaced by one or more atoms independently chosen from nitrogen, oxygen, or sulfur atoms. Any nitrogen atom maybe optionally oxidized or quaternized, and any sulfur atom maybe optionally oxidized. One or more of the carbons may be oxidized to C(═O). Unless otherwise specified, the heterocycloalkenyl ring may be attached at any carbon atom or heteroatom that results in a stable structure and, if substituted, may be substituted at any suitable carbon atom or heteroatom which results in a stable structure. Examples of heterocycloalkenyl groups are saturated heterocycloalkenyl groups containing from two to nine carbon atoms and one to four heteroatoms. Generally, 5-7 membered heterocycloalkenyl groups contain 3-6 carbon atoms and 1-2 heteroatoms independently selected from the group consisting of N, S, S═O, S(═O)₂, and O. Examples of heterocycloalkenyl groups include but are not limited to dihydropyran, dihydrofuran, and the like. “Carbon-attached heterocycloalkenyl” refers to a heterocycloalkenyl group which is bound via a constituent carbon atom. Terms such as “heterocycloalkenyloxy”, “heterocycloalkenylthio”, “heterocycloalkenylamino” refer to heterocycloalkenyl groups linked to a second group via an oxygen, sulfur, or nitrogen atom, respectively.

The term “acyl” refers to a monovalent radical of the formula —C(═O)-alkyl and —C(═O)-cycloalkyl, i.e., an alkyl or cycloalkyl group linked to a second group via carbonyl group C(═O), wherein said alkyl maybe further substituted with cycloalkyl, aryl, or heteroaryl. Examples of acyl groups include —C(═O)Me (acetyl), —C(═O)CH₂-cyclopropyl (cyclopropylacetyl), —C(═O)CH₂Ph (phenylacetyl), and the like.

The term “aryl” refers to 6-10 membered mono- or polycyclic aromatic carbocycles, for example, phenyl and naphthyl. Unless otherwise specified, the aryl ring may be attached at any carbon atom that results in a stable structure and, if substituted, may be substituted at any suitable carbon atom which results in a stable structure. The term “aryl” refers to non-substituted aryls and aryls optionally substituted with one or more substituents. Aryl maybe abbreviated “Ar”. It should be understood that any combination term using an “ar” or “aryl” prefix refers to analogs according to the above definition of “aryl” including the number of carbon atoms. For example, terms such as “aryloxy”, “arylthio”, and “arylamino” refer to aryl groups linked to a second group via an oxygen, sulfur, or nitrogen atom, respectively.

The term “arylalkyl” refers to alkyl groups substituted with an aryl group and refers to aryl groups linked to another group via an sp³ carbon atom. Examples include benzyl, α-methylbenzyl and phenethyl groups.

The term “heteroaryl” refers to a stable 5-8 membered monocyclic or 8-11 membered bicyclic aromatic heterocycle radical. In one embodiment the monocyclic groups are 5 or 6 membered. Each heteroaryl contains 1-10 carbon atoms and from 1 to 5 heteroatoms independently chosen from nitrogen, oxygen and sulfur, wherein any sulfur heteroatom may optionally be oxidized and any nitrogen heteroatom may optionally be oxidized or quaternized. Unless otherwise specified, the heteroaryl ring may be attached at any suitable heteroatom or carbon atom that results in a stable structure and, if substituted, may be substituted at any suitable heteroatom or carbon atom which results in a stable structure. The term “heteroaryl” includes heteroaryl groups that are non-substituted or those optionally substituted. Generally, heteroaryl groups containing 1-9 carbon atoms and 1-4 heteroatoms independently selected from the group N, S, S═O, S(═O)₂, and O. It should be understood that any combination term using “heteroaryl” refers to analogs according to the above definition of heteroaryl including the numbers of carbon and heteroatoms. Examples of “heteroaryl” include but are not limited to radicals such as furanyl, thienyl, pyrrolyl, oxazolyl, thiazolyl, imidazolyl, pyrazolyl, isoxazolyl, isothiazolyl, oxadiazolyl, triazolyl, tetrazolyl, thiadiazolyl, pyridinyl, pyridazinyl, pyrimidinyl, pyrazinyl, indolizinyl, indolyl, isoindolyl, benzofuranyl, benzothienyl, indazolyl, benzimidazolyl, benzothiazolyl, benzoxazolyl, benzisoxazolyl, benzisothiazolyl, purinyl, quinolizinyl, quinolinyl, isoquinolinyl, cinnolinyl, phthalazinyl, quinazolinyl, quinoxalinyl, naphthyridinyl, pteridinyl, carbazolyl, acridinyl, phenazinyl, phenothiazinyl and phenoxazinyl. Terms such as “heteroaryloxy”, “heteroarylthio”, “heteroarylamino” refer to heteroaryl groups linked to a second group via an oxygen, sulfur, or nitrogen atom, respectively.

The term “heteroarylalkyl” refers to alkyl groups substituted with a heteroaryl group and refers to a heteroaryl that is linked to a second group via an sp³ carbon atom. Examples include 2- 3- and 4-pyridylmethyl and 2-(2-pyridyl)ethyl groups.

The term “amino” group is —NH₂. Alkylamino and dialkylamino groups, for example, include the groups —NHR²¹ and —NR²¹R²² wherein each R²¹ and R²² are independently substituted or unsubstituted C₁₋₁₀alkyl groups. Example of such groups include —NHMe, —NHEt, —NHcyclohexyl, —NHCH₂phenyl, —N(Me)₂ and the like. Useful dialkylamino groups include any of the above-mentioned C₁₋₁₀alkyl groups, each substituted or unsubstituted. Also, a substituted amino group may include for example —NHMe, —NHEt, —NHcyclohexyl, —NHCH₂phenyl, —N(Me)₂ and the like, and —NHCOMe, —NHCOEt, —NHCONHMe and the like. Useful alkylamino and dialkylamino are —NHR²¹ and —NR²¹R²², wherein R²¹ and R²² are C₁₋₁₀alkyl groups, each unsubstituted or substituted by any of the previously mentioned dialkyl amino groups. In one aspect, R²¹ and R²² are independently C₁₋₁₀alkyl groups. A dialkylamino group, such as —NR²¹R²² includes the group wherein R²¹ and R²² are combined with the nitrogen to which they attach to form a ring, such as a 3-membered, 4-membered, 5-membered or 6-membered ring and their fused, bicyclic analogs, each of which may be further substituted as defined herein. Non-exclusive examples of such rings may include aziridines, pyrrolidines, piperidines, piperazines, morpholines and the like. In certain variations of the nitrogen containing ring, the ring may comprise one or more double bonds and may be fully or partially unsaturated.

All of the groups defined above may be optionally substituted as defined below.

The terms “optional” or “optionally” mean that the subsequently described event or circumstances may or may not occur, and that the description includes instances where the event or circumstance occurs and instances in which it does not. For example, “optionally substituted aryl” means that the aryl radical may or may not be substituted and that the description includes both substituted aryl radicals and aryl radicals having no substitution. In one aspect, optional substitution is 0-5 substitutions of the groups described below. Exemplary optional substituents include one or more of the following groups: halogen, C₁-C₁₀ alkyl, C₃-C₆ cycloalkyl, C₂-C₁₀ alkenyl, C₄-C₆ cycloalkenyl, C₂-C₆ alkynyl, nitro, cyano, hydroxyl, C₁-C₆ alkoxy, C₃-C₆ cycloalkoxy, amido, amino, C₁-C₆ alkylamino (for example, —NHMe- or —N(Me)₂), C₁-C₆ carbamoyl, C₁-C₆ carboxy, C₁-C₆ carbonyl, C₁-C₆ acyl, thiol, C₁-C₆ alkylthio, and C₁-C₆ carboxylic acid. Such substituents can further be substituted with optionally selected groups to form a stable structure.

As used herein “solvate” refers to a complex of variable stoichiometry formed by a solute (e.g. a compound of formula (I) or a salt, ester or prodrug thereof) and a solvent. Such solvents for the purpose of the invention may not interfere with the biological activity of the solute. Examples of suitable solvents include water, methanol, ethanol and acetic acid. Generally the solvent used is a pharmaceutically acceptable solvent. Examples of suitable pharmaceutically acceptable solvents include water, ethanol and acetic acid. Generally the solvent used is water.

“Isomers” mean any compound with an identical molecular formula but having a difference in the nature or sequence of bonding or arrangement of the atoms in space. Examples of such isomers include, for example, E- and Z-isomers of double bonds, enantiomers, and diastereomers. Compounds of the present invention depicting a bond with a straight line or “squiggly line” representation that is attached to a double bond, unless specifically noted otherwise, is intended to encompass a single isomer and/or both isomers of the double bond as shown below mean any compound with an identical molecular formula but having a difference in the nature or sequence of bonding or arrangement of the atoms in space.

As used herein “allosteric modulator” of α7 nAChR refers to a compound that binds allosterically to α7 nAChR, thereby increasing (positive allosteric modulator) or decreasing (negative allosteric modulator) the agonist-evoked response in cells.

As used herein “disorders amenable to modulation of α7 nAChRs” refers to neurodegenerative diseases, senile dementias, schizophrenia, Alzheimer's disease, learning, cognition and attention deficits, memory loss, Lewy Body dementia, attention-deficit disorder, attention deficit hyperactivity disorder, anxiety, mania, manic depression, Parkinson's disease, Huntington's disease, depression, amyotrophic lateral sclerosis, brain inflammation, cognitive deficit due to traumatic brain injury (“TBI”) and Tourette's syndrome. In addition, such disorders include immune system disorders such as, but not limited to, type I diabetes, multiple schlerosis, and rheumatoid arthritis. “Disorders amenable to modulation of α7 nAChRs” also include pain, inflammation, septic shock, ulcerative colitis, Crohn's disease, irritable bowel syndrome, and jet lag. Also included are autism spectrum disorders, inflammation, and mild cognitive impairment.

As used herein “a cognitive disorder related to learning or memory” refers to mild cognitive impairment, age related cognitive decline, senile dementia and Alzheimer's disease.

### Formulations

Compounds of the invention are administered orally in a total daily dose of about 0.01 mg/kg/dose to about 100 mg/kg/dose, alternately from about 0.1 mg/kg/dose to about 10 mg/kg/dose. The use of time-release preparations to control the rate of release of the active ingredient may be employed. The dose may be administered in as many divided doses as is convenient. When other methods are used (e.g. intravenous administration), compounds are administered to the affected tissue at a rate from 0.05 to 10 mg/kg/hour, alternately from 0.1 to 1 mg/kg/hour. Such rates are easily maintained when these compounds are intravenously administered as discussed below.

For the purposes of this invention, the compounds may be administered by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters. Oral administration is generally employed.

Pharmaceutical compositions containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax maybe employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents.

Syrups and elixirs may be formulated with sweetening agents, such as glycerol, sorbitol or sucrose. Such formulations may also contain a demulcent, a preservative, a flavoring or a coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions. The pharmaceutical composition can be prepared to provide easily measurable amounts for administration. For example, an aqueous solution intended for intravenous infusion should contain from about 3 to 330 μg of the active ingredient per milliliter of solution in order that infusion of a suitable volume at a rate of about 30 mL/hr can occur.

As noted above, formulations of the present invention suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach. This is particularly advantageous with the compounds of Formulae I-VII when such compounds are susceptible to acid hydrolysis.

Formulations suitable for topical administration in the mouth include lozenges comprising the active ingredient in a flavored base, usually sucrose and acacia or tragacanth; pastilles comprising the active ingredient in an inert base such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Injection solutions and suspensions may be prepared from sterile powders, granules and tablets of the kind previously described.

Suitable unit dosage formulations are those containing a daily dose or unit, daily sub-dose, or an appropriate fraction thereof, of a compound of Formulae I-VII.

It will be understood, however, that the specific dose level for any particular patient will depend on a variety of factors including the activity of the specific compound employed; the age, body weight, general health, sex and diet of the individual being treated; the time and route of administration; the rate of excretion; other drugs which have previously been administered; and the severity of the particular disease undergoing therapy, as is well understood by those skilled in the art.

In one embodiment of this invention, X¹ is NH—R¹, X² is C—R², X³ is C—R³, X⁴ is C—R⁴, X¹¹ is C—R¹¹, X¹² is N—R¹², X¹³ is C—R¹³, X¹⁴ is C—R¹⁴, X¹⁵ is C—R¹⁵ and X¹⁶ is C—R¹⁶, with the remaining groups as defined for Formula I such that representative allosteric α7 nAChR modulators of this invention include compounds having the structure of Formula II:

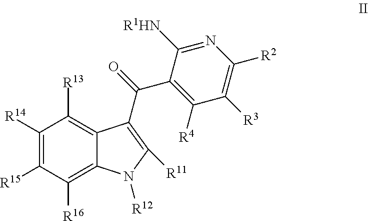

and pharmaceutically acceptable salts, solvates, and prodrugs thereof.

In another embodiment, such compounds are selected from those wherein
R¹ is selected from the group consisting of C₁₋₈ alkyl and C₁₋₈ haloalkyl, each optionally substituted; or

R¹ is selected from the group consisting of aryl, heteroaryl, arylalkyl, heteroarylalkyl, C₃₋₈ cycloalkyl, cycloalkenyl, heterocycloalkyl and heterocycloalkenyl, each optionally substituted; and

R², R³, R⁴, R¹¹, R¹³, R¹⁴, R¹⁵ and R¹⁶ are each independently selected from the group consisting of hydrogen, halogen, cyano, amino, C₁₋₈ alkyl, C₁₋₈ haloalkyl, C₃₋₈ cycloalkyl, cycloalkenyl, heterocycloalkyl heterocycloalkenyl, C₁₋₈ alkoxy, C₁₋₈ haloalkoxy, C₃₋₈ cycloalkoxy, C₁₋₈ alkamino, dialkylamino, C₃₋₈ cycloalkamino, cycloalkenylamino, heterocycloalkylamino, heterocycloalkenylamino, C₁₋₈ alkthio, C₁₋₈ haloalkthio, and C₃₋₈ cycloalkthio; and

R¹² is selected from the group consisting of hydrogen, C₁₋₈ alkyl, C₃₋₈ cycloalkyl, and C₁₋₈ haloalkyl,

and pharmaceutically acceptable salts and prodrugs thereof.

In another embodiment, such compounds are selected from those wherein
R¹ is selected from the group consisting of arylalkyl and heteroarylalkyl, each optionally substituted;

R¹² is selected from the group consisting of hydrogen and C₁₋₈ alkyl;

R¹³ is hydrogen; and

R¹⁴, R¹⁵ and R¹⁶ are each independently selected from the group consisting of hydrogen, halogen, C₁₋₈ alkyl, and C₁₋₈ haloalkyl; and pharmaceutically acceptable salts, solvates, and prodrugs thereof.

In another embodiment, such compounds are selected from those wherein
R¹ is optionally substituted benzyl; and

R², R³, R⁴, R¹¹, R¹³, R¹⁴, R¹⁵ and R¹⁶ are each independently selected from the group consisting of hydrogen, halogen, cyano, amino, C₁₋₈ alkyl, C₁₋₈ haloalkyl, C₃₋₈ cycloalkyl, cycloalkenyl, heterocycloalkyl heterocycloalkenyl, C₁₋₈ alkoxy, C₁₋₈ haloalkoxy, C₃₋₈ cycloalkoxy, C₁₋₈ alkamino, dialkylamino, C₃₋₈ cycloalkamino, cycloalkenylamino, heterocycloalkylamino, heterocycloalkenylamino, C₁₋₈ alkthio, C₁₋₈ haloalkthio, and C₃₋₈ cycloalkthio; and

R¹² is selected from the group consisting of hydrogen, C₁₋₈ alkyl, C₃₋₈ cycloalkyl, and C₁₋₈ haloalkyl, and pharmaceutically acceptable salts and prodrugs thereof.

In another embodiment, such compounds are selected from those wherein
R¹ is optionally substituted benzyl;

R¹² is selected from the group consisting of hydrogen and C₁₋₈ alkyl;

R¹³ is hydrogen; and

R¹⁴, R¹⁵ and R¹⁶ are each independently selected from the group consisting of hydrogen, halogen, C₁₋₈ alkyl, and C₁₋₈ haloalkyl; and pharmaceutically acceptable salts, solvates, and prodrugs thereof.

In another embodiment, such compounds are selected from those wherein

R¹ is an optionally substituted arylalkyl;

R¹² is selected from the group consisting of hydrogen and C₁₋₈ alkyl;

R¹³ is hydrogen;

R¹⁴ and R¹⁵ are each independently selected from the group consisting of hydrogen and halogen; and

R¹⁶ is hydrogen; and pharmaceutically acceptable salts, solvates, and prodrugs thereof.

In another embodiment, such compounds are selected from those wherein

R¹ is an optionally substituted benzyl;

R¹² is selected from the group consisting of hydrogen and C₁₋₈ alkyl;

R¹³ is hydrogen;

R¹⁴ and R¹⁵ are each independently selected from the group consisting of hydrogen and halogen; and

R¹⁶ is hydrogen; and pharmaceutically acceptable salts, solvates, and prodrugs thereof.

In one embodiment of this invention, X¹ is O—R¹, X² is C—R², X³ is C—R³, X⁴ is C—R⁴, X¹¹ is C—R¹¹, X¹² is N—R¹², X¹³ is C—R¹³, X¹⁴ is C—R¹⁴, X¹⁵ is C—R¹⁵ and X¹⁶ is C—R¹⁶, with the remaining groups as defined for Formula I such that representative allosteric α7 nAChR modulators of this invention include compounds having the structure of Formula III:

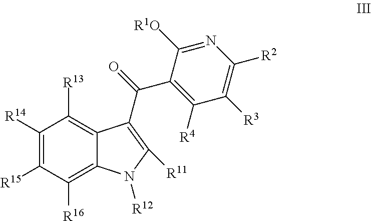

and pharmaceutically acceptable salts, solvates, and prodrugs thereof.

In another embodiment of this invention, X¹ is NH—R¹, X² is C—R², X³ is C—R³, X⁴ is C—R⁴, X¹¹ is N, X¹² is N—R¹², X¹³ is C—R¹³, X¹⁴ is C—R¹⁴, X¹⁵ is C—R¹⁵ and X¹⁶ is C—R¹⁶, with the remaining groups as defined for Formula I such that representative allosteric α7 nAChR modulators of this invention include compounds having the structure of Formula IV:

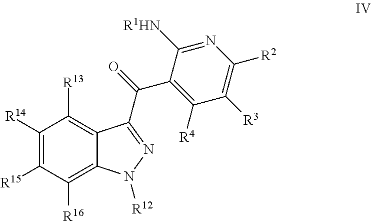

and pharmaceutically acceptable salts, solvates, and prodrugs thereof.

In another embodiment, such compounds are selected from those wherein
R¹ is selected from the group consisting of C₁₋₈ alkyl and C₁₋₈ haloalkyl, each optionally substituted; or

R¹ is selected from the group consisting of aryl, heteroaryl, arylalkyl, heteroarylalkyl, C₃₋₈ cycloalkyl, cycloalkenyl, heterocycloalkyl and heterocycloalkenyl, each optionally substituted; and

R², R³, R⁴, R¹³, R¹⁴, R¹⁵ and R¹⁶ are each independently selected from the group consisting of hydrogen, halogen, cyano, amino, C₁₋₈ alkyl, C₁₋₈ haloalkyl, C₃₋₈ cycloalkyl, cycloalkenyl, heterocycloalkyl heterocycloalkenyl, C₁₋₈ alkoxy, C₁₋₈ haloalkoxy, C₃₋₈ cycloalkoxy, C₁₋₈ alkamino, dialkylamino, C₃₋₈ cycloalkamino, cycloalkenylamino, heterocycloalkylamino, heterocycloalkenylamino, C₁₋₈ alkthio, C₁₋₈ haloalkthio, and C₃₋₈ cycloalkthio; and

R¹² is selected from the group consisting of hydrogen, C₁₋₈ alkyl, C₃₋₈ cycloalkyl, and C₁₋₈ haloalkyl, and pharmaceutically acceptable salts and prodrugs thereof.

In another embodiment, such compounds are selected from those wherein
R¹ is optionally substituted benzyl; and

R², R³, R⁴, R¹³, R¹⁴, R¹⁵ and R¹⁶ are each independently selected from the group consisting of hydrogen, halogen, cyano, amino, C₁₋₈ alkyl, C₁₋₈ haloalkyl, C₃₋₈ cycloalkyl, cycloalkenyl, heterocycloalkyl heterocycloalkenyl, C₁₋₈ alkoxy, C₁₋₈ haloalkoxy, C₃₋₈ cycloalkoxy, C₁₋₈ alkamino, dialkylamino, C₃₋₈ cycloalkamino, cycloalkenylamino, heterocycloalkylamino, heterocycloalkenylamino, C₁₋₈ alkthio, C₁₋₈ haloalkthio, and C₃₋₈ cycloalkthio; and R¹² is selected from the group consisting of hydrogen, C₁₋₈ alkyl, C₃₋₈ cycloalkyl, and C₁₋₈ haloalkyl, and pharmaceutically acceptable salts and prodrugs thereof.

In another embodiment, such compounds are selected from those wherein
R¹ is selected from the group consisting of arylalkyl and heteroarylalkyl, each optionally substituted;

R¹² is selected from the group consisting of hydrogen and C₁₋₈ alkyl;

R¹³ is hydrogen; and

R¹⁴, R¹⁵ and R¹⁶ are each independently selected from the group consisting of hydrogen, halogen, C₁₋₈ alkyl, and C₁₋₈ haloalkyl; and pharmaceutically acceptable salts, solvates, and prodrugs thereof.

In another embodiment, such compounds are selected from those wherein
R¹ is optionally substituted benzyl;

R¹² is selected from the group consisting of hydrogen and C₁₋₈ alkyl;

R¹³ is hydrogen; and

R¹⁴, R¹⁵ and R¹⁶ are each independently selected from the group consisting of hydrogen, halogen, C₁₋₈ alkyl, and C₁₋₈ haloalkyl; and pharmaceutically acceptable salts, solvates, and prodrugs thereof.

In another embodiment, such compounds are selected from those wherein

R¹ is an optionally substituted arylalkyl;

R¹² is selected from the group consisting of hydrogen and C₁₋₈ alkyl;

R¹³ is hydrogen;

R¹⁴ and R¹⁵ are each independently selected from the group consisting of hydrogen and halogen; and

R¹⁶ is hydrogen; and pharmaceutically acceptable salts, solvates, and prodrugs thereof.

In another embodiment, such compounds are selected from those wherein

R¹ is an optionally substituted benzyl;

R¹² is selected from the group consisting of hydrogen and C₁₋₈ alkyl; R¹³ is hydrogen;

R¹⁴ and R¹⁵ are each independently selected from the group consisting of hydrogen and halogen; and R¹⁶ is hydrogen; and pharmaceutically acceptable salts, solvates, and prodrugs thereof.

In still another embodiment of this invention, X¹ is NH—R, X² is C—R², X³ is N, X⁴ is C—R⁴, X¹ is C—R¹¹, X¹² is N—R², X¹³ is C—R³, X¹⁴ is C—R¹⁴, X¹⁵ is C—R¹⁵ and X¹⁶ is C—R¹⁶, with the remaining groups as defined for Formula I such that representative allosteric α7 nAChR modulators of this invention include compounds having the structure of Formula V:

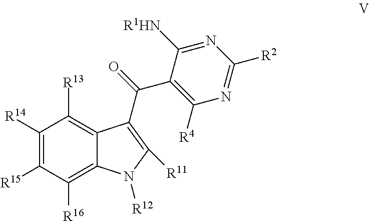

and pharmaceutically acceptable salts, solvates, and prodrugs thereof.

In another embodiment, such compounds are selected from those wherein
R¹ is selected from the group consisting of C₁₋₈ alkyl and C₁₋₈ haloalkyl, each optionally substituted; or

R¹ is selected from the group consisting of aryl, heteroaryl, arylalkyl, heteroarylalkyl, C₃₋₈ cycloalkyl, cycloalkenyl, heterocycloalkyl and heterocycloalkenyl, each optionally substituted; and

R², R⁴, R¹³, R¹⁴, R¹⁵ and R¹⁶ are each independently selected from the group consisting of hydrogen, halogen, cyano, amino, C₁₋₈ alkyl, C₁₋₈ haloalkyl, C₃₋₈ cycloalkyl, cycloalkenyl, heterocycloalkyl heterocycloalkenyl, C₁₋₈ alkoxy, C₁₋₈ haloalkoxy, C₃₋₈ cycloalkoxy, C₁₋₈ alkamino, dialkylamino, C₃₋₈ cycloalkamino, cycloalkenylamino, heterocycloalkylamino, heterocycloalkenylamino, C₁₋₈ alkthio, C₁₋₈ haloalkthio, and C₃₋₈ cycloalkthio; and

R¹² is selected from the group consisting of hydrogen, C₁₋₈ alkyl, C₃₋₈ cycloalkyl, and C₁₋₈ haloalkyl, and pharmaceutically acceptable salts and prodrugs thereof.

In another embodiment, such compounds are selected from those wherein
R¹ is optionally substituted benzyl; and

R², R⁴, R¹³, R¹⁴, R and R¹⁶ are each independently selected from the group consisting of hydrogen, halogen, cyano, amino, C₁₋₈ alkyl, C₁₋₈ haloalkyl, C₃₋₈ cycloalkyl, cycloalkenyl, heterocycloalkyl heterocycloalkenyl, C₁₋₈ alkoxy, C₁₋₈haloalkoxy, C₃₋₈ cycloalkoxy, C₁₋₈ alkamino, dialkylamino, C₃₋₈ cycloalkamino, cycloalkenylamino, heterocycloalkylamino, heterocycloalkenylamino, C₁₋₈ alkthio, C₁₋₈ haloalkthio, and C₃₋₈ cycloalkthio; and

R¹² is selected from the group consisting of hydrogen, C₁₋₈ alkyl, C₃₋₈ cycloalkyl, and C₁₋₈ haloalkyl, and pharmaceutically acceptable salts and prodrugs thereof.

In another embodiment, such compounds are selected from those wherein
R¹ is selected from the group consisting of arylalkyl and heteroarylalkyl, each optionally substituted;
R¹² is selected from the group consisting of hydrogen and C₁₋₈ alkyl;
R¹³ is hydrogen;
R¹⁴, R¹⁵ and R¹⁶ are each independently selected from the group consisting of hydrogen, halogen, C₁₋₈ alkyl, and C₁₋₈ haloalkyl;
and pharmaceutically acceptable salts, solvates, and prodrugs thereof.

In another embodiment, such compounds are selected from those wherein
R¹ is optionally substituted benzyl;
R¹² is selected from the group consisting of hydrogen and C₁₋₈ alkyl;
R¹³ is hydrogen;
R¹⁴, R¹⁵ and R¹⁶ are each independently selected from the group consisting of hydrogen, halogen, C₁₋₈ alkyl, and C₁₋₈ haloalkyl;
and pharmaceutically acceptable salts, solvates, and prodrugs thereof. In another embodiment, such compounds are selected from those wherein
R¹ is an optionally substituted arylalkyl;
R¹² is selected from the group consisting of hydrogen and C₁₋₈ alkyl;
R¹³ is hydrogen;
R¹⁴ and R¹⁵ are each independently selected from the group consisting of hydrogen and halogen;
R¹⁶ is hydrogen;
   and pharmaceutically acceptable salts, solvates, and prodrugs thereof.

In another embodiment, such compounds are selected from those wherein
R¹ is an optionally substituted benzyl;
R¹² is selected from the group consisting of hydrogen and C₁₋₈ alkyl;
R¹³ is hydrogen;
R¹⁴ and R¹⁵ are each independently selected from the group consisting of hydrogen and halogen;
R¹⁶ is hydrogen;
and pharmaceutically acceptable salts, solvates, and prodrugs thereof.

In still another embodiment of this invention, X¹ is NH—R, X² is N, X³ is C—R³, X⁴ is C—R⁴, X¹¹ is C—R¹¹, X¹² is N—R¹², X¹³ is C—R³, X¹⁴ is C—R¹⁴, X¹⁵ is C—R¹⁵ and X¹⁶ is C—R¹⁶, with the remaining groups as defined for Formula I such that representative allosteric α7 nAChR modulators of this invention include compounds having the structure of Formula VI:

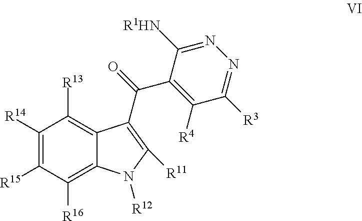

and pharmaceutically acceptable salts, solvates, and prodrugs thereof.

In another embodiment, such compounds are selected from those wherein
R¹ is selected from the group consisting of C₁₋₈ alkyl and C₁₋₈ haloalkyl, each optionally substituted; or

R¹ is selected from the group consisting of aryl, heteroaryl, arylalkyl, heteroarylalkyl, C₃₋₈ cycloalkyl, cycloalkenyl, heterocycloalkyl and heterocycloalkenyl, each optionally substituted; and

R³, R⁴, R¹³, R¹⁴, R¹⁵ and R¹⁶ are each independently selected from the group consisting of hydrogen, halogen, cyano, amino, C₁₋₈ alkyl, C₁₋₈ haloalkyl, C₃₋₈ cycloalkyl, cycloalkenyl, heterocycloalkyl heterocycloalkenyl, C₁₋₈ alkoxy, C₁₋₈ haloalkoxy, C₃₋₈ cycloalkoxy, C₁₋₈ alkamino, dialkylamino, C₃₋₈ cycloalkamino, cycloalkenylamino, heterocycloalkylamino, heterocycloalkenylamino, C₁₋₈ alkthio, C₁₋₈ haloalkthio, and C₃₋₈ cycloalkthio; and

R¹² is selected from the group consisting of hydrogen, C₁₋₈ alkyl, C₃₋₈ cycloalkyl, and C₁₋₈ haloalkyl, and pharmaceutically acceptable salts and prodrugs thereof.

In another embodiment, such compounds are selected from those wherein
R¹ is optionally substituted benzyl; and

R³, R⁴, R³, R¹⁴, R¹⁵ and R¹⁶ are each independently selected from the group consisting of hydrogen, halogen, cyano, amino, C₁₋₈ alkyl, C₁₋₈ haloalkyl, C₃₋₈ cycloalkyl, cycloalkenyl, heterocycloalkyl heterocycloalkenyl, C₁₋₈ alkoxy, C₁₋₈ haloalkoxy, C₃₋₈ cycloalkoxy, C₁₋₈ alkamino, dialkylamino, C₃₋₈ cycloalkamino, cycloalkenylamino, heterocycloalkylamino, heterocycloalkenylamino, C₁₋₈ alkthio, C₁₋₈ haloalkthio, and C₃₋₈ cycloalkthio; and

R¹² is selected from the group consisting of hydrogen, C₁₋₈ alkyl, C₃₋₈ cycloalkyl, and C₁₋₈ haloalkyl, and pharmaceutically acceptable salts and prodrugs thereof.

In another embodiment, such compounds are selected from those wherein

R¹ is selected from the group consisting of arylalkyl and heteroarylalkyl, each optionally substituted;

R¹² is selected from the group consisting of hydrogen and C₁₋₈ alkyl;

R¹³ is hydrogen; and

R¹⁴, R¹⁵ and R¹⁶ are each independently selected from the group consisting of hydrogen, halogen, C₁₋₈ alkyl, and C₁₋₈ haloalkyl; and pharmaceutically acceptable salts, solvates, and prodrugs thereof.

In another embodiment, such compounds are selected from those wherein
R¹ is optionally substituted benzyl;
R¹² is selected from the group consisting of hydrogen and C₁₋₈ alkyl;
R¹³ is hydrogen; and
R¹⁴, R¹⁵ and R¹⁶ are each independently selected from the group consisting of hydrogen, halogen, C₁₋₈ alkyl, and C₁₋₈ haloalkyl;
and pharmaceutically acceptable salts, solvates, and prodrugs thereof. In another embodiment, such compounds are selected from those wherein
R¹ is an optionally substituted arylalkyl;
R¹² is selected from the group consisting of hydrogen and C₁₋₈ alkyl;
R¹³ is hydrogen;
R¹⁴ and R¹⁵ are each independently selected from the group consisting of hydrogen and halogen;
R¹⁶ is hydrogen;
and pharmaceutically acceptable salts, solvates, and prodrugs thereof.

In another embodiment, such compounds are selected from those wherein
R¹ is an optionally substituted benzyl;
R¹² is selected from the group consisting of hydrogen and C₁₋₈ alkyl;
R¹³ is hydrogen;
R¹⁴ and R¹⁵ are each independently selected from the group consisting of hydrogen and halogen;
R¹⁶ is hydrogen;
and pharmaceutically acceptable salts, solvates, and prodrugs thereof.

In still another embodiment of this invention, X¹ is NH—R, X² is C—R², X³ is C—R³, X⁴ is N, X¹¹ is C—R¹¹, X¹² is N—R¹², X¹³ is C—R¹³, X¹⁴ is C—R¹⁴, X¹⁵ is C—R¹⁵ and X¹⁶ is C—R¹⁶, with the remaining groups as defined for Formula I such that representative allosteric α7 nAChR modulators of this invention include compounds having the structure of Formula VII:

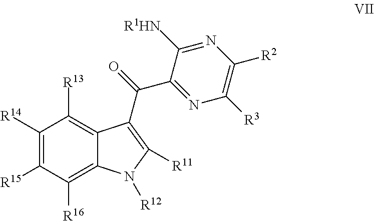

and pharmaceutically acceptable salts, solvates, and prodrugs thereof.

In another embodiment, such compounds are selected from those wherein
R¹ is selected from the group consisting of C₁₋₈ alkyl and C₁₋₈ haloalkyl, each optionally substituted; or

R¹ is selected from the group consisting of aryl, heteroaryl, arylalkyl, heteroarylalkyl, C₃₋₈ cycloalkyl, cycloalkenyl, heterocycloalkyl and heterocycloalkenyl, each optionally substituted; and

R², R³, R¹³, R¹⁴, R¹⁵ and R¹⁶ are each independently selected from the group consisting of hydrogen, halogen, cyano, amino, C₁₋₈ alkyl, C₁₋₈ haloalkyl, C₃₋₈ cycloalkyl, cycloalkenyl, heterocycloalkyl heterocycloalkenyl, C₁₋₈ alkoxy, C₁₋₈ haloalkoxy, C₃₋₈ cycloalkoxy, C₁₋₈ alkamino, dialkylamino, C₃₋₈ cycloalkamino, cycloalkenylamino, heterocycloalkylamino, heterocycloalkenylamino, C₁₋₈ alkthio, C₁₋₈ haloalkthio, and C₃₋₈ cycloalkthio; and

R¹² is selected from the group consisting of hydrogen, C₁₋₈ alkyl, C₃₋₈ cycloalkyl, and C₁₋₈ haloalkyl, and pharmaceutically acceptable salts and prodrugs thereof.

In another embodiment, such compounds are selected from those wherein
R¹ is optionally substituted benzyl; and

R², R³, R¹³, R¹⁴, R¹⁵ and R¹⁶ are each independently selected from the group consisting of hydrogen, halogen, cyano, amino, C₁₋₈ alkyl, C₁₋₈ haloalkyl, C₃₋₈ cycloalkyl, cycloalkenyl, heterocycloalkyl heterocycloalkenyl, C₁₋₈ alkoxy, C₁₋₈ haloalkoxy, C₃₋₈ cycloalkoxy, C₁₋₈ alkamino, dialkylamino, C₃₋₈ cycloalkamino, cycloalkenylamino, heterocycloalkylamino, heterocycloalkenylamino, C₁₋₈alkthio, C₁₋₈ haloalkthio, and C₃₋₈ cycloalkthio; and

R¹² is selected from the group consisting of hydrogen, C₁₋₈ alkyl, C₃₋₈ cycloalkyl, and C₁₋₈ haloalkyl, and pharmaceutically acceptable salts and prodrugs thereof.

In another embodiment, such compounds are selected from those wherein
R¹ is selected from the group consisting of arylalkyl and heteroarylalkyl, each optionally substituted;
R¹² is selected from the group consisting of hydrogen and C₁₋₈ alkyl;
R¹³ is hydrogen; and
R¹⁴, R¹⁵ and R¹⁶ are each independently selected from the group consisting of hydrogen, halogen, C₁₋₈ alkyl, and C₁₋₈ haloalkyl;
and pharmaceutically acceptable salts, solvates, and prodrugs thereof.

In another embodiment, such compounds are selected from those wherein
R¹ is optionally substituted benzyl;
R¹² is selected from the group consisting of hydrogen and C₁₋₈ alkyl;
R¹³ is hydrogen; and
R¹⁴, R¹⁵ and R¹⁶ are each independently selected from the group consisting of hydrogen, halogen, C₁₋₈ alkyl, and C₁₋₈ haloalkyl;
and pharmaceutically acceptable salts, solvates, and prodrugs thereof.

In another embodiment, such compounds are selected from those wherein
R¹ is an optionally substituted arylalkyl;
R¹² is selected from the group consisting of hydrogen and C₁₋₈ alkyl;
R¹³ is hydrogen;
R¹⁴ and R¹⁵ are each independently selected from the group consisting of hydrogen and halogen;
R¹⁶ is hydrogen;
and pharmaceutically acceptable salts, solvates, and prodrugs thereof.

In another embodiment, such compounds are selected from those wherein
R¹ is an optionally substituted benzyl;
R¹² is selected from the group consisting of hydrogen and C₁₋₈ alkyl;
R¹³ is hydrogen;
R¹⁴ and R¹⁵ are each independently selected from the group consisting of hydrogen and halogen;
R¹⁶ is hydrogen;
and pharmaceutically acceptable salts, solvates, and prodrugs thereof.

In one aspect, compounds of Formula I include:
(6-chloro-1H-indol-3-yl)[2-(cyclopentylamino)pyridine-3-yl]methanone (Compound 1);
[2-(benzylamino)pyridine-3-yl](6-chloro-1H-indol-3-yl)methanone (Compound 2),
(6-chloro-1H-indol-3-yl)[2-(pyridine-2-ylmethylamino)pyridine-3-yl]methanone (Compound 3);
(6-chloro-1H-indol-3-yl)[2-(phenethylamino)pyridine-3-yl]methanone (Compound 4);
[2-(benzylamino)pyridine-3-yl](6-fluoro-1H-indol-3-yl)methanone (Compound 5);
(6-chloro-1H-indol-3-yl)[2-(pyridine-4-ylmethylamino)pyridine-3-yl]methanone (Compound 6);
[2-(benzylamino)pyridine-3-yl](5-chloro-1H-indol-3-yl)methanone (Compound 7);
(5-chloro-1H-indol-3-yl)[2-(phenethylamino)pyridine-3-yl]methanone (Compound 8);
(5-chloro-1H-indol-3-yl)[2-(phenylamino)pyridine-3-yl]methanone (Compound 9);
(6-chloro-1H-indol-3-yl)[2-[(4-fluorophenyl)amino]pyridine-3-yl]]methanone (Compound 10);
(6-chloro-1H-indol-3-yl)[2-(phenylamino)pyridine-3-yl]methanone (Compound 11);
[2-(benzylamino)pyridine-3-yl](7-chloro-1H-indol-3-yl)methanone (Compound 12);
(6-chloro-1H-indol-3-yl)[2-(4-fluorobenzylamino)pyridine-3-yl]methanone (Compound 13);
(6-chloro-1H-indol-3-yl)[2-(4-methoxybenzylamino)pyridine-3-yl]methanone (Compound 14);
(6-chloro-1H-indol-3-yl)[2-(3,4-difluorobenzylamino)pyridine-3-yl]methanone (Compound 15);
(6-chloro-1H-indol-3-yl)[2-(2,4-difluorobenzylamino)pyridine-3-yl]methanone (Compound 16);
(6-chloro-1H-indol-3-yl)[2-(4-chlorobenzylamino)pyridine-3-yl]methanone (Compound 17);
(6-chloro-1H-indol-3-yl)[2-(4-methylbenzylamino)pyridine-3-yl]methanone (Compound 18);
(6-chloro-1H-indol-3-yl)[2-(cyclohexylmethylamino)pyridine-3-yl]methanone (Compound 19);
(6-chloro-1H-indol-3-yl)[2-(cyclopropylmethylamino)pyridine-3-yl]methanone (Compound 20);
(6-chloro-1H-indol-3-yl)[2-(propylamino)pyridine-3-yl]methanone (Compound 21);
[2-(benzylamino)pyridine-3-yl](6-chloro-1H-indazol-3-yl)methanone (Compound 22);
[2-(benzylamino)pyridine-3-yl](1H-indazol-3-yl)methanone (Compound 23);
[2-(benzylamino)pyridine-3-yl](6-chloro-1-methyl-1H-indol-3-yl)methanone (Compound 24);
[2-(benzylamino)-6-methylpyridine-3-yl](6-chloro-1H-indol-3-yl)methanone (Compound 25);
[2-(tetrahydro-2H-pyran-4-ylamino)pyridine-3-yl](6-chloro-1H-indol-3-yl)methanone (Compound 26);
(6-chloro-1H-indol-3-yl)[2-[(4-fluorobenzyl)amino]pyrazin-3-yl]methanone (Compound 27) and
(6-chloro-1H-indol-3-yl)[6-chloro-[3-(4-fluorobenzyl)amino]pyridazin-4-yl]methanone (Compound 28) and pharmaceutically acceptable salts, solvates, and prodrugs thereof.

In another aspect, there is provided pharmaceutical compositions comprising a compound of Formulae I-VII, and pharmaceutically acceptable salts, solvates, and prodrugs thereof.

In yet another aspect there is provided a method for the treatment of disorders amenable to modulation of α7 nAChR comprising administering to a patient in need of such treatment a compound of Formulae I-VII or a pharmaceutically acceptable salt, solvate, or prodrug thereof. In one embodiment, the disorder is a neurodegenerative disorder. In another embodiment, the disorder is a senile dementia. In another embodiment, the disorder is schizophrenia. In another embodiment, the disorder is a cognition deficit disorder. In another embodiment, the disorder is Alzheimer's disease. In another embodiment, the disorder is a learning, cognition and attention deficits disorder, memory loss, Lewy Body dementia, attention-deficit disorder, attention deficit hyperactivity disorder, anxiety, mania, manic depression, Parkinson's disease, Huntington's disease, depression, amyotrophic lateral sclerosis, brain inflammation, cognitive deficit due to traumatic brain injury, and Tourette's syndrome. In another embodiment, the disorder is pain, inflammation, septic shock, ulcerative colitis, Crohn's disease and irritable bowel syndrome. In yet another aspect, there is provided a method of treating inflammation. In yet another aspect there is provided a method for the treatment of diabetes and jetlag. In another embodiment, compounds of the invention are useful in tobacco cessation treatment and in treating immune system disorders. In another embodiment, the disorder is depression and the treatment comprising the administration of a compound of Formulae I-VII or a pharmaceutically acceptable salt or prodrug thereof and the administration of an SSRI drug, a drug that augments 5-HT release or blocks 5-HT reuptake. In yet another embodiment, the disorder is an immune disorder.

In another aspect, there is provided a method for the treatment of disorders related to learning and memory such as mild cognitive impairment, age related cognitive decline, senile dementia, and Alzheimer's disease comprising administering to a patient in need of such treatment a compound of Formulae I-VII or a pharmaceutically acceptable salt or prodrug thereof. In one embodiment the treatment of such disorders is achieved via modulation of mono and divalent cation conductance through the site mediating the action of a compound of Formulae I-VII or a pharmaceutically acceptable salt or prodrug thereof.

In another aspect, there is a provided a method for the treatment of immune system disorders, Fragile X, autism spectrum disorder, Angelman's syndrome, Tett Syndrome, Prader Willi syndrome and Down's syndrome by administering to a patient in need thereof a compound of Formulae I-VII, a pharmaceutically acceptable salt, solvate, or prodrug thereof.

For use in medicine, the salts of the compounds of Formulae I-VII will be pharmaceutically acceptable salts. Other salts may, however, be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulfuric acid, methanesulfonic acid, fumaric acid, maleic acid, succinic acid, acetic acid, benzoic acid, oxalic acid, citric acid, tartaric acid, or phosphoric acid. Furthermore, where the compound comprises an acidic moiety, suitable pharmaceutically acceptable salts thereof may include alkali metal salts, e.g. sodium or potassium salts; alkaline earth metal salts, e.g. calcium or magnesium salts; and salts formed with suitable organic ligands, e.g. quaternary ammonium salts. Standard methods for the preparation of pharmaceutically acceptable salts and their formulations are well known in the art, and are disclosed in various references, including for example, “Remington: The Science and Practice of Pharmacy”, A. Gennaro, ed., 20th edition, Lippincott, Williams & Wilkins, Philadelphia, Pa.

The present invention includes prodrugs of the compounds of Formulae I-VII above. In general, such prodrugs will be functional derivatives of the compounds of Formulae I-VII that are readily convertible in vivo into the required compound of Formulae I-VII. Conventional procedures for the selection and preparation of suitable prodrug derivatives are described, for example, in Design of Prodrugs, ed. H. Bundgaard, Elsevier, 1985. Such prodrugs include but are not limited to ester prodrugs from alcohols and acids, phosphate prodrugs of alcohols, and N-oxide derivatives of heteroaryl moieties. The prodrug can be formulation to achieve a goal of improved chemical stability, improved patient acceptance and compliance, improved bioavailability, prolonged duration of action, improved organ selectivity, improved formulation (e.g., increased hydrosolubility), and/or decreased side effects (e.g., toxicity).

Where the compounds of the present invention have at least one asymmetric center, they may accordingly exist as enantiomers. Where the compounds possess two or more asymmetric centers, they may additionally exist as diastereoisomers. It is to be understood that all such stereoisomers and mixtures thereof in any proportion are encompassed within the scope of the present invention. Where the compounds possess geometrical isomers, all such isomers and mixtures thereof in any proportion are encompassed within the scope of the present invention.

Tautomers of the compounds of the invention are encompassed by the present application. Thus, for example, a carbonyl includes its hydroxyl tautomer.

### EXAMPLES

Standard procedures and chemical transformation and related methods are well known to one skilled in the art, and such methods and procedures have been described, for example, in standard references such as Fiesers' Reagents for Organic Synthesis, John Wiley and Sons, New York, N.Y., 2002; Organic Reactions, vols. 1-83, John Wiley and Sons, New York, N.Y., 2006; March J. and Smith M.: Advanced Organic Chemistry, 6th ed., John Wiley and Sons, New York, N.Y.; and Larock R. C.: Comprehensive Organic Transformations, Wiley-VCH Publishers, New York, 1999. All texts and references cited herein are incorporated by reference in their entirety.

Reactions using compounds having functional groups may be performed on compounds with functional groups that may be protected. A “protected” compound or derivatives means derivatives of a compound where one or more reactive site or sites or functional groups are blocked with protecting groups. Protected derivatives are useful in the preparation of the compounds of the present invention or in themselves; the protected derivatives may be the biologically active agent. An example of a comprehensive text listing suitable protecting groups may be found in T. W. Greene, Protecting Groups in Organic Synthesis, 3rd edition, John Wiley & Sons, Inc. 1999.

Compounds of Formula II can be prepared as shown in Scheme 1, starting with commercially available indoles of Formula A. Treatment of an appropriately substituted indole of Formula A with diethylaluminum chloride in dichloromethane at 0° C. followed by addition of 2-chloronicotinoyl chloride, slow warming to ambient temperature and workup provides the corresponding indole 3-carbonyl B (cf Okauchi, Org. Lett. 2000, 10, 1485-1487). Further reaction with an appropriate amine leads to molecules of Formula II (cf Giannouli, J. Med. Chem., 2007, 50, 1716-1719).

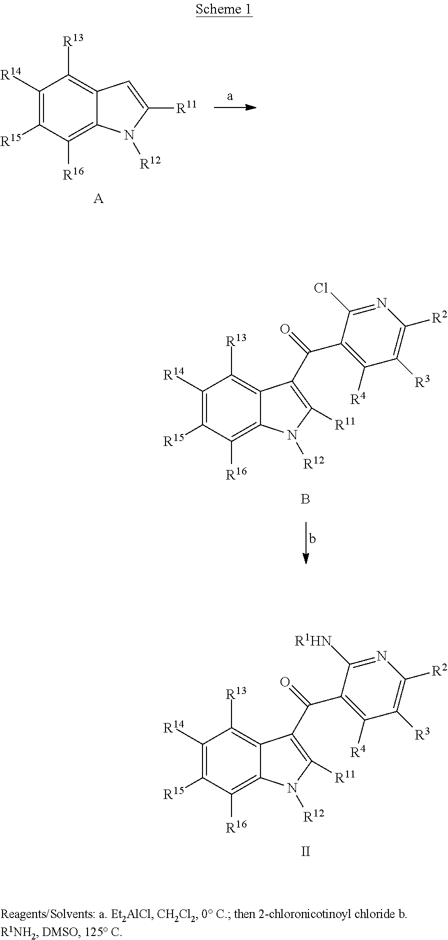

Compounds of Formula IV can be prepared as shown in Scheme 2, starting with compounds of Formula C. Reaction with a hydrazine provides compounds of Formula D. Further reaction with bromine leads to compounds of Formula E. Treatment of E with base, such as n-BuLi and t-BuLi at −78° C. and subsequent reaction with an appropriately substituted nicotinoyl chloride gave intermediate F. Reaction with an amine HNR₅R₆ then gives compounds of Formula IV.

The indole-3-carbonyl B can also be prepared as in Scheme 3. Reaction of an appropriately substituted 2-nitrotoluene G with N,N-dimethylformamide dimethylacetal (DMFDMA) in DMF at reflux forms the enamine H. (Batcho, A. D. and Leimgruber, W. Org. Synth. 1985, 63, 214) Reaction with an appropriately substituted 2-chloronicotinoyl chloride in the presence of a trialkylamine or 1,4-diazabicyclo[2.2.2]octane (DABCO) affords the enaminone I (Shahrisa, A. et al. J. Heterocyclic Chem. 2009, 46, 273). Reduction of the nitro group (Zn metal in MeOH/THF or Pt/C doped with V in MeOH/H₂) then affords the indole-3-carbonyl B (WO 2010/051373; WO 2011/137342; Bryan, C. et al. Org. Synth. 2009, 86, 36 and Baumeister, P. et al. Catal. Lett. 1997, 49, 219). The reaction can also be carried out with other appropriately substituted heteroarylcarbonyl chlorides such as pyrazinecarbonyl chlorides and pyridazinecarbonyl chlorides.

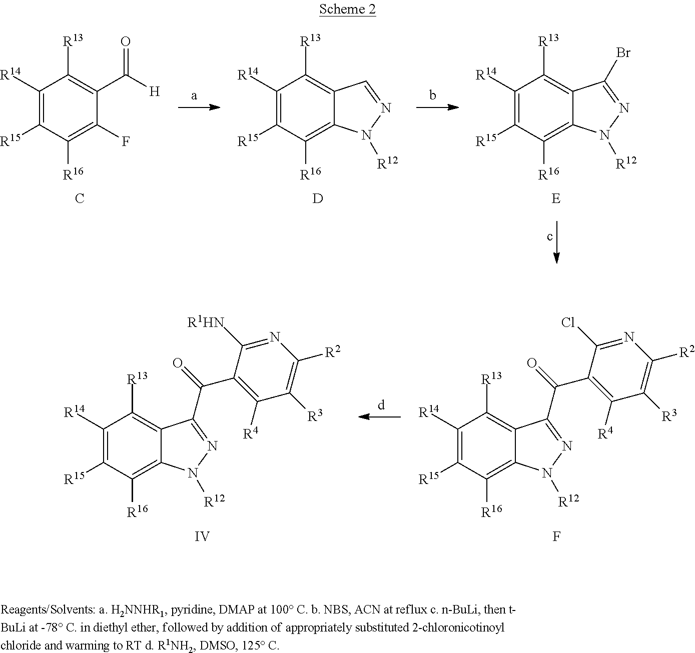

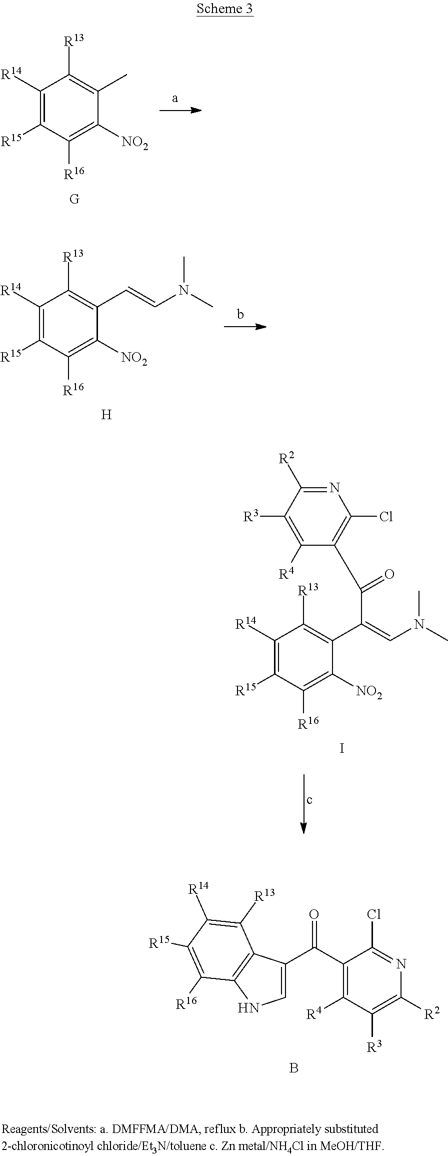

Oocyte Electrophysiology:

Individual compounds were tested for modulation of submaximal nicotine-evoked currents at α7 nAChRs using oocytes expressing human receptors. For each oocyte, the maximal nicotine-evoked currents were determined in response to 3 μM nicotine. All other currents were scaled to this value. The concentration of nicotine was adjusted to evoke a fractional current of approximately 0.05 (5% of max, or “EC₅”), and this concentration of nicotine was used to generate EC₅ control currents. Increasing concentrations of test compounds were applied to oocytes alone (pretreatment) and then in combination with the EC₅ concentration of nicotine (co-application). This protocol allowed measurement of both direct effects of test compounds on α7 nAChRs, and modulatory effects of compounds on nicotine-evoked responses. mRNA was prepared and stored using conventional techniques from cDNA clones encoding the human nicotinic receptor subunits. Preparation, micro-injection and maintenance of oocytes were performed as reported in detail previously (Whittemore et al., Mol. Pharmacol. 50: 1364-1375, 1996). Individual oocytes were injected with 5-50 ng of each subunit mRNA. For multiple subunit combinations, the mRNA ratios are: (1) a4132 and a3134 nAChRs (a 1:1 mixture); Following injections, oocytes were maintained at 16-17° C. in Barth's medium. Two-electrode voltage clamp recordings were made 3-14 days following mRNA injections at a holding voltage of −70 mV unless specified. The nicotinic recordings were done in Ca⁺⁺-free Ringer solution (mM: NaCl, 115; KCl, 2; BaCl₂, 1.8; HEPES, 5; pH 7.4) to limit Ca⁺⁺-activated chloride and muscarinic currents. Drug and wash solutions were applied using a microcapillary “linear array” (Hawkinson et al., Mol. Pharmacol. 49: 897-906, 1996) in order to allow rapid application of agonists. Currents were recorded on a chart recorder and/or PC-based computer for subsequent analysis. Test compounds were made up in DMSO over a concentration range of 0.001-10 mM and diluted 1000-3000-fold into the appropriate saline just prior to testing (final [DMSO]<0.1%). The concentration-dependence of modulation was analyzed using GraphPad “Prism” curve-fitting software.

Positive allosteric modulators can also be assayed by imaging of calcium flux through α7 nAChR transiently expressed in a cell line, including HEK-293 and cell cultered neurons. (see for example international published application WO 2006/071184)

### Example 1

### (6-Chloro-1H-indol-3-yl)(2-(cyclopentylamino)pyridine-3-yl)methanone

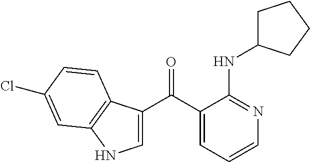 [SMILES: O=C(c1cccnc1NC1CCCC1)c1c[nH]c2cc(Cl)ccc12]

### (6-Chloro-1H-indol-3-yl)(2-chloropyridin-3-yl)methanone

A solution of 6-chloroindole (2.5 g, 16.5 mmol) in 50 mL of CH₂Cl₂ at 0° C. was treated with diethyl aluminumchloride (4.55 mL, 36.3 mmol) added dropwise while maintaining the temperature at 0° C. After stirring for 1 h, the solution was treated with 2-chloronicotinoyl chloride (solid, in one portion 2.9 g, 16.5 mmol). The resulting mixture was allowed to warm to RT, and was quenched with a 10% aqueous solution of Rochelle's Salt. The biphasic mixture was filtered through celite, more CH₂Cl₂ was added, the layers separated and the aqueous discarded. The CH₂Cl₂ extract was dried over MgSO₄ and concentrated in vacuo to afford an off white solid (3.1 g) that was used without further purification.

### (6-Chloro-1H-indol-3-yl)[2-(cyclopentylamino)pyridine-3-yl]methanone

Solid (6-chloro-1H-indol-3-yl)(2-chloropyridine-3-yl)methanone (0.150 g) was dissolved in DMSO (1.5 mL). Excess cyclopentylamine was added and the mixture was heated to 100° C. for several hours. The mixture was then cooled to RT and purified by RPHLPC to give an off white solid (0.095 g). MS 340 (M+H)⁺.

The following compounds were prepared by using the procedure described above:

### [2-(Benzylamino)pyridine-3-yl](6-chloro-1H-indol-3-yl)methanone

[2-(Benzylamino)-pyridine-3-yl](6-chloro-1H-indol-3-yl)methanone was prepared using the procedure for (6-chloro-1H-indol-3-yl)[2-(cyclopentylamino)pyridine-3-yl]methanone except that cyclopentylamine was replaced with benzylamine. MS 362 (M+H)⁺

### (6-Chloro-1H-indol-3-yl)[2-(pyridine-2-ylmethylamino)pyridine-3-yl]methanone

(6-Chloro-1H-indol-3-yl)[2-(pyridine-2-ylmethylamino)pyridine-3-yl]methanone was prepared using the procedure for (6-chloro-1H-indol-3-yl)[2-(cyclopentylamino)pyridine-3-yl]methanone except that cyclopentylamine was replaced with 2-aminomethylpyridine. MS 363 (M+H)⁺

### (6-Chloro-1H-indol-3-yl)[2-(phenethylamino)pyridine-3-yl]methanone

(6-Chloro-1H-indol-3-yl)[2-(phenethylamino)pyridine-3-yl]methanone was prepared using the procedure for (6-chloro-1H-indol-3-yl)[2-(cyclopentylamino)pyridine-3-yl]methanone except that cyclopentylamine was replaced with phenethylamine. MS 376 (M+H)⁺

### [2-(Benzylamino)pyridine-3-yl](6-fluoro-1H-indol-3-yl)methanone

[2-(Benzyl-amino)pyridine-3-yl](6-fluoro-1H-indol-3-yl)methanone was prepared using the procedure for (6-chloro-1H-indol-3-yl)[2-(cyclopentylamino)pyridine-3-yl]methanone except that 6-chloroindole was replaced with 6-fluoroindole and cyclopentylamine was replaced with benzylamine. MS 346 (M+H)⁺

### (6-Chloro-1H-indol-3-yl)[2-(pyridin-4-ylmethylamino)pyridine-3-yl]methanone

(6-Chloro-1H-indol-3-yl)[2-(pyridin-4-ylmethylamino)pyridine-3-yl]methanone was prepared using the procedure for (6-chloro-1H-indol-3-yl)[2-(cyclopentylamino)pyridine-3-yl]methanone except that cyclopentylamine was replaced with 4-aminomethylpyridine. MS 363 (M+H)⁺

### [2-(Benzylamino)pyridine-3-yl](5-chloro-1H-indol-3-yl)methanone

[2-(Benzyl-amino)pyridine-3-yl](5-chloro-1H-indol-3-yl)methanone was prepared using the procedure for (6-chloro-1H-indol-3-yl)[2-(cyclopentylamino)pyridine-3-yl]methanone except that 6-chloroindole was replaced with 5-chloroindole and cyclopentylamine was replaced with benzylamine. MS 362 (M+H)⁺

### (5-Chloro-1H-indol-3-yl)[2-(phenethylamino)pyridine-3-yl]methanone

(5-Chloro-1H-indol-3-yl)[2-(phenethylamino)pyridine-3-yl]methanone was prepared using the procedure for (6-chloro-1H-indol-3-yl)[2-(cyclopentylamino)pyridine-3-yl]methanone except that 6-chloroindole was replaced with 5-chloroindole and cyclopentylamine was replaced with phenethylamine. MS 376 (M+H)⁺

### (5-Chloro-1H-indol-3-yl)[(2-phenylamino)pyridine-3-yl]methanone

(5-Chloro-1H-indol-3-yl)[(2-phenylamino)pyridine-3-yl]methanone was prepared using the procedure for (6-chloro-1H-indol-3-yl)[2-(cyclopentylamino)pyridine-3-yl]methanone except that 6-chloroindole was replaced with 5-chloroindole and cyclopentylamine was replaced with aniline. MS 348 (M+H)⁺

### (6-Chloro-1H-indol-3-yl)[2-(4-fluorophenylamino)pyridine-3-yl]methanone

(6-Chloro-1H-indol-3-yl)[2-(4-fluorophenylamino)pyridine-3-yl]methanone was prepared using the procedure for (6-chloro-1H-indol-3-yl)[2-(cyclopentylamino)pyridine-3-yl]methanone except that cyclopentylamine was replaced with 4-fluoroaniline. MS 366 (M+H)⁺

### (6-Chloro-1H-indol-3-yl)[(2-phenylamino)pyridine-3-yl]methanone

(6-Chloro-1H-indol-3-yl)[(2-phenylamino)pyridine-3-yl]methanone was prepared using the procedure for (6-chloro-1H-indol-3-yl)[2-(cyclopentylamino)pyridine-3-yl]methanone except that cyclopentylamine was replaced with aniline. MS 348 (M+H)⁺

### [2-(Benzylamino)pyridine-3-yl](7-chloro-1H-indol-3-yl)methanone

[2-(Benzyl-amino)pyridine-3-yl](7-chloro-1H-indol-3-yl)methanone was prepared using the procedure for (6-chloro-1H-indol-3-yl)(2-(cyclopentylamino)pyridine-3-yl)methanone except that 6-chloroindole was replaced with 7-chloroindole and cyclopentylamine was replaced with benzylamine. MS 362 (M+H)⁺

### (6-Chloro-1H-indol-3-yl)[2-(4-fluorobenzylamino)pyridine-3-yl]methanone

(6-Chloro-1H-indol-3-yl)[2-(4-fluorobenzylamino)pyridine-3-yl]methanone was prepared using the procedure for (6-chloro-1H-indol-3-yl)[2-(cyclopentylamino)pyridine-3-yl]methanone except that cyclopentylamine was replaced with 4-fluorobenzylamine. MS 380 (M+H)⁺

### (6-Chloro-1H-indol-3-yl)[2-(4-methoxybenzylamino)pyridine-3-yl]methanone

(6-Chloro-1H-indol-3-yl)[2-(4-methoxybenzylamino)pyridine-3-yl]methanone was prepared using the procedure for (6-chloro-1H-indol-3-yl)[2-(cyclopentylamino)pyridine-3-yl]methanone except that cyclopentylamine was replaced with 4-methoxybenzylamine. MS 392 (M+H)⁺

### (6-Chloro-1H-indol-3-yl)[2-(3,4-difluorobenzylamino)pyridine-3-yl]methanone

(6-Chloro-1H-indol-3-yl)[2-(3,4-difluorobenzylamino)pyridine-3-yl]methanone was prepared using the procedure for (6-chloro-1H-indol-3-yl)[2-(cyclopentylamino)pyridine-3-yl]methanone except that cyclopentylamine was replaced with 3,4-difluorobenzylamine. MS 398 (M+H)⁺

### (6-Chloro-1H-indol-3-yl)[2-(2,4-difluorobenzylamino)pyridine-3-yl]methanone

(6-Chloro-1H-indol-3-yl)[2-(2,4-difluorobenzylamino)pyridine-3-yl]methanone was prepared using the procedure for (6-chloro-1H-indol-3-yl)[2-(cyclopentylamino)pyridine-3-yl]methanone except that cyclopentylamine was replaced with 2,4-difluorobenzylamine. MS 398 (M+H)⁺

### (6-Chloro-1H-indol-3-yl)[2-(4-chlorobenzylamino)pyridine-3-yl]methanone

(6-Chloro-1H-indol-3-yl)[2-(4-chlorobenzylamino)pyridine-3-yl]methanone was prepared using the procedure for (6-chloro-1H-indol-3-yl)[2-(cyclopentylamino)pyridine-3-yl]methanone except that cyclopentylamine was replaced with 4-chlorobenzylamine. MS 396 (M+H)⁺

### (6-Chloro-1H-indol-3-yl)(2-(4-methylbenzylamino)pyridine-3-yl)methanone

(6-Chloro-1H-indol-3-yl)[2-(4-methylbenzylamino)pyridine-3-yl]methanone was prepared using the procedure for (6-chloro-1H-indol-3-yl)[2-(cyclopentylamino)pyridine-3-yl]methanone except that cyclopentylamine was replaced with 4-methylbenzylamine. MS 376 (M+H)⁺

### (6-Chloro-1H-indol-3-yl)[2-(cyclohexylmethylamino)pyridine-3-yl]methanone

(6-Chloro-1H-indol-3-yl)[2-(cyclohexylmethylamino)pyridine-3-yl]methanone was prepared using the procedure for (6-chloro-1H-indol-3-yl)[2-(cyclopentylamino)pyridine-3-yl]methanone except that cyclopentylamine was replaced with cyclohexylmethylamine. MS 368 (M+H)⁺

### (6-Chloro-1H-indol-3-yl)[2-(cyclopropylmethylamino)pyridine-3-yl]methanone

(6-Chloro-1H-indol-3-yl)[2-(cyclopropylmethylamino)pyridine-3-yl]methanone was prepared using the procedure for (6-chloro-1H-indol-3-yl)[2-(cyclopentylamino)pyridine-3-yl]methanone except that cyclopentylamine was replaced with cyclopropylmethylamine. MS 326 (M+H)⁺

### (6-Chloro-1H-indol-3-yl)[2-(propylamino)pyridine-3-yl]methanone

(6-Chloro-1H-indol-3-yl)[2-(propylamino)pyridine-3-yl]methanone was prepared using the procedure for (6-chloro-1H-indol-3-yl)[2-(cyclopentylamino)pyridine-3-yl]methanone except that cyclopentylamine was replaced with propylamine. MS 314 (M+H)⁺[2-(Benzylamino)-6-methylpyridine-3-yl](6-chloro-1H-indol-3-yl)methanone. [2-(Benzylamino)-6-methylpyridine-3-yl](6-chloro-1H-indol-3-yl)methanone was prepared using the procedure for (6-chloro-1H-indol-3-yl)[2-(cyclopentylamino)pyridine-3-yl]methanone except that 2-chloronicotinoyl chloride was replaced with 2-chloro-6-methylnicotinoyl chloride and cyclopentylamine was replaced with benzylamine. MS 376 (M+H)⁺

### [2-(Tetrahydro-2H-furan-4-ylamino)pyridine-3-yl](6-chloro-1H-indol-3-yl)methanone

[2-(Tetrahydro-2H-furan-4-ylamino)-pyridine-3-yl](6-chloro-1H-indol-3-yl)methanone was prepared using the procedure for (6-chloro-1H-indol-3-yl)[2-(cyclopentylamino)pyridine-3-yl]methanone except that cyclopentylamine was replaced with 4-aminotetrahydro-2H-pyran. MS 356 (M+H)⁺

### Example 2

### [2-(Benzylamino)pyridine-3-yl](6-chloro-1H-indazol-3-yl)methanone

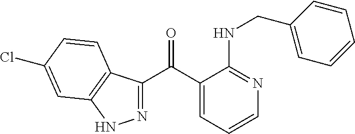 [SMILES: O=C(c1cccnc1NCc1ccccc1)c1n[nH]c2cc(Cl)ccc12]

### 6-Chloroindazole

A solution of 4-chloro-2-fluorobenzaldehyde (5.0 g, 31.6 mmol) in 12 mL of pyridine was treated with hydrazine hydrate (10 eq., 10 mL) and DMAP (3.85 g, 31.6 mmol) and the mixture heated to 100° C. for several hours. The mixture was allowed to cool to RT and was diluted with EtOAc and washed several times with dilute acid. The EtOAc solution was dried over MgSO₄ and concentrated in vacuo to give 6-chloroindazole as an off white solid (3.86 g).

### (6-Chloro-1H-indazol-3-yl)(2-chloropyridine-3-yl)methanone

6-Chloro-indazole (3.86 g, 25.4 mmol) was added to 45 mL of 20% aq. NaOH solution, and reacted with neat Br₂ (0.85 mL, 16.3 mmol) for several hours at RT. The reaction was neutralized with the product, 3-bromo-6-chloroindazole, precipitating as a white solid (4.2 g). A solution of 3-bromo-6-chloroindazole (0.46 g, 2 mmol) in diethyl ether (6 mL) was cooled to −78° C. and n-BuLi (1.6 M in hexanes; 1.25 mL, 2 mmol) was added dropwise. After the addition was complete, t-BuLi (1.7 M in hexanes; 2.36 mL, 4 mmol) was added dropwise. The solution was allowed to stir 15 minutes at −78° C., after which 2-chloronicotinoyl chloride (0.35 g, 2.0 mmol) was added as a solid. The solution was allowed to warm to RT, and was then quenched with 1N HCl and extracted with EtOAc. The EtOAc extract was concentrated in vacuo, and the residue purified by RPHPLC to give 100 mg of (6-chloro-1H-indazol-3-yl)(2-chloropyridine-3-yl)methanone as an off white solid.

### [2-(Benzylamino)pyridine-3-yl](6-chloro-1H-indazol-3-yl)methanone

(6-Chloro-1H-indazol-3-yl)(2-chloropyridine-3-yl)methanone (0.10 g, 0.34 mmol) was dissolved in 3 mL of DMSO and benzylamine (0.074 mL, 0.68 mmol) was added. The solution was heated to 130° C. for 1 h, and then allowed to cool to RT. The product solution was purified by chromatography to give the title compound, [(2-(benzylamino)pyridine-3-yl](6-chloro-1H-indazol-3-yl)methanone (0.015 g), as a white solid. MS 363 (M+H)⁺

The following compounds were prepared by using the procedure described above:

### [2-(Benzylamino)pyridine-3-yl](1H-indazol-3-yl)methanone

[2-(Benzylamino)pyridine-3-yl](1H-indazol-3-yl)methanone was prepared using the method described for [(2-(benzylamino)pyridine-3-yl](6-chloro-1H-indazol-3-yl)methanone except that 6-chloroindazole was replaced with indazole. MS 329 (M+H)⁺

### Example 3

### [2-(Benzylamino)pyridine-3-yl](6-chloro-1-methyl-1H-indol-3-yl)methanone

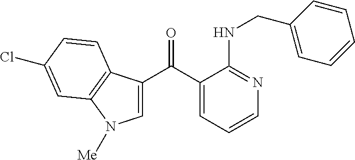 [SMILES: Cn1cc(C(=O)c2cccnc2NCc2ccccc2)c2ccc(Cl)cc21]

[2-(Benzylamino)pyridine-3-yl](6-chloro-1H-indol-3-yl)methanone (0.10 g, 0.28 mmol) was dissolved in 3 mL of DMF. Sodium hydride (60% dispersion; 0.013 g, 0.34 mmol) was added in one portion. To this was added methyl iodide (0.019 mL, 0.31 mmol) and the solution was allowed to stir for 30 min. The solution was quenched with dilute acid and extracted with EtOAc. The EtOAc layer was separated and concentrated in vacuo, and the residue was purified by RPHPLC to give (0.05 g) of [2-(benzylamino)pyridine-3-yl](6-chloro-1-methyl-1H-indol-3-yl)methanone. MS 376 (M+H)⁺

### Example 4

### (6-Chloro-1H-indol-3-yl)[2-[(4-fluorobenzyl)amino]pyrazin-3-yl]methanone

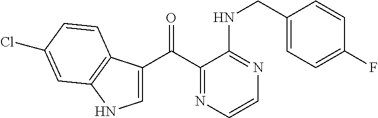 [SMILES: O=C(c1nccnc1NCc1ccc(F)cc1)c1c[nH]c2cc(Cl)ccc12]

### 3-Amino-2-pyrazinecarboxylic acid

Methyl 3-amino-2-pyrazinecarboxylate (Aldrich; 2.08 g, 13.6 mmol) suspended in MeOH (40 mL) under N₂ was treated with a 1N aq. NaOH solution (30 mL). Reaction warmed and was stirred at rt for 50 min. The MeOH was partly removed in vacuo (45 mL of liquid removed). The resulting mixture was cooled in an ice/water bath and a 1N aq. HCl solution was added (70 mL). The mixture initially gave a solution and as more acid was added, a ppt formed. The solid was filtered and washed with water (3×10 mL) to give 1.48 g of product after drying in vacuo.

Ref: Synth. Comm. 2010, 40, 2988 and WO 2011/143129

### 3-Hydroxy-2-pyrazinecarboxylic acid

A suspension of 3-amino-2-pyrazinecarboxylic acid (1.42 g, 10.2 mmol) in water (11.5 mL) was treated with a 3.75 M H₂SO₄ solution (stock made from 80 mL water and 20 mL conc. H₂SO₄; 11.5 mL. Most of the solid dissolved. Mixture placed in an oil bath at 50° C. (11:10 am-11:20 am) until a soln formed. The rxn was allowed to cool to rt and was then treated at 10-15° C. (ppt formed) with a solution of NaNO₂ (840 mg, 12.2 mmol) in 3.3 mL of water added dropwise over 35 min. The reaction was stirred at rt for 30 min, then placed in an oil bath at 50° C. and the temperature was increased to 100° C. and heated at reflux for 30 min. Once at rt, the mixture was filtered and the solid was washed with 5 mL of water to give the crude product. This material was suspended in 5 mL water and treated with a sat. aq. NaHCO₃ soln (10 mL). The resulting hazy solution was filtered and the filter was washed with water (2×5 mL). The brown solution was then treated with a 1N aq. HCl solution (13 mL). The ppt was collected and washed with an aq. 1N HCl soln to give 1.03 g of the acid.

Ref: WO 2011/143129

### 3-Chloro-2-pyrazinecarbonyl chloride

3-Hydroxy-2-pyrazinecarboxylic acid (455 mg, 3.27 mmol) in POCl₃ (6 mL) was treated with 3 drops of pyridine and heated to reflux for 2 h. Once at rt, the reaction was conc. to dryness. The residue was dissolved in toluene and conc. in vacuo and then triturated with hexanes (4×10 mL). The hexanes washes decanted, combined and conc. This material in toluene (10 mL) containing 1 drop of DMF was treated with neat SOCl₂ (2 mL) and heated at reflux for 3 h. The mixture was decanted and conc. in vacuo. The residue was dissolved in toluene and conc. to give 355 mg of a purple liquid that solidified on standing.

### (6-Chloro-1H-indol-3-yl)(2-chloropyrazin-3-yl)methanone

6-Chloroindole (Alfa-Aesar; 223 mg, 1.47 mmol) in CH₂Cl₂ (7 mL) cooled in an ice/water bath a neat Et₂AlCl (MW 120.56, d 0.961; 0.3 mL) was added via syringe. The reaction was stirred cold for 30 min. A soln of 3-chloro-2-pyrazinecarbonyl chloride (331 mg, 1.88 mmol) in 3 mL of CH₂Cl₂ was added dropwise via syringe over 10 min. The dark mixture was stirred cold for 2 h, then overnight at rt. The reaction was then diluted with 30 mL of CH₂Cl₂ and quenched with a pH 6-7 2M potassium phosphate buffer (10 mL). The resulting mixture filtered through Celite and the Celite was washed with 40 mL of CH₂Cl₂. The separated organic layer was washed with water (50 mL) and brine (30 mL). After drying (MgSO₄), the mixture was filtered and conc. to give 280 mg of crude product. Trituration with CH₂Cl₂ gave 54 mg of the product as a dark brown solid.

### (6-Chloro-1H-indol-3-yl)[2-[(4-fluorobenzyl)amino]pyrazin-3-yl]methanone

(6-Chloro-1H-indol-3-yl)(2-chloropyrazin-3-yl)methanone (48 mg, 0.16 mmol) in DMSO (0.6 mL) was treated with neat 4-fluorobenzylamine (Acros; 95 μL, 104 gm, 0.83 mmol). The dark reaction was placed in an oil bath at 120° C. for 2 h. Once at rt, the reaction was added to ice/water and the resulting ppt was collected and partitioned between CH₂Cl₂ and water. The aq. layer was washed twice with CH₂Cl₂ and the pooled organic layers washed with water (3 x) and brine. The solution was dried (MgSO₄), filtered and conc. to give 55 mg of crude product. Column chromatography (2.5% MeOH/CH₂Cl₂) gave 27 mg of the title compound as a light yellow solid. ¹H NMR (400 MHz, CDCl₃) δ 9.50 (br s, 1H), 8.88 (d, 1H, J=3.1 Hz), 8.68 (br s, 1H), 8.46 (d, 1H, J=8.6 Hz), 8.24 (d, 1H, J=2.1 Hz), 7.90 (d, 1H, J=2.3 Hz), 7.42 (d, 1H, J=1.2 Hz), 7.37 (dd, 2H, J=8.8, 5.2 Hz), 7.28 (dd, 1H, J=8.4, 2.0 Hz), 7.02 (t, 2H, J=8.7 Hz), 4.76 (d, 2H, J=5.8 Hz).

### Example 5

### (6-Chloro-1H-indol-3-yl)[6-chloro-[3-(4-fluorobenzyl)amino]pyridazin-4-yl]methanone

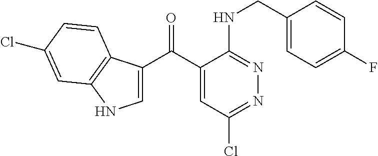 [SMILES: O=C(c1cc(Cl)nnc1NCc1ccc(F)cc1)c1c[nH]c2cc(Cl)ccc12]

### 3,6-Dichloropyridazine-4-carbonyl chloride

3,6-Dichloropyridazine-4-carboxylic acid (Aldrich; 1.13 g, 5.86 mmol) in toluene (20 mL) containing 2 drops of DMF was treated with neat SOCl₂ (4 mL). The mixture heated to reflux for 3 h and then allowed to cool. The resulting red-orange mixture was decanted and conc. in vacuo. The residue was redissolved in toluene and conc to give 1.22 g of the product.

### (6-Chloro-1H-indol-3-yl)[3,6-dichloropyridazin-4-yl)methanone

A solution of 6-chloroindole (Alfa-Aesar; 632 mg, 4.17 mmol) in CH₂Cl₂ (20 mL) was cooled in an ice/water bath and neat Et₂AlCl (Aldrich, MW 120.56, d 0.961; 0.85 mL, 817 mg, 6.77 mmol) was added dropwise via syringe. The reaction was stirred cold for 30 min and then treated with a solution of 3,6-dichloropyridazine-4-carbonyl chloride (1.22 g, 5.77 mmol) in CH₂Cl₂ (4 mL) added via syringe over 20 min. The reaction was stirred cold for 2 h and allowed to warm to rt overnight. The dark mixture was cooled in an ice/water bath and treated with a 2M pH 7 phosphate buffer. The reaction was diluted with CH₂Cl₂ and filtered to remove 291 mg of a yellow-brown solid. The CH₂Cl₂ washes were pooled and conc. to give an additional 582 mg of solid. The solids were combined and dissolved as much as possible in 5% MeOH/CH₂Cl₂. Column chromatography (5% MeOH/CH₂Cl₂) gave 242 mg of the product.

### (6-Chloro-1H-indol-3-yl)[6-chloro-[3-(4-fluorobenzyl)amino]pyridazin-4-yl]-methanone

(6-Chloro-1H-indol-3-yl)[3,6-dichloropyridazin-4-yl)methanone (97 mg, 0.30 mmol) in DMSO (1 mL) was treated with neat 4-fluorobenzylamine (5 eq, 170 μL, 186 mg, 1.50 mmol) and heated at 120° C. for 2 h. Once at rt, the reaction added with stirring to ice/water. The resulting ppt was collected and washed with water to give a yellow-brown solid. Flash chromatography (2.5% MeOH/CH₂Cl₂) gave 14 mg of the title compound as a bright yellow solid. TOF MS ES⁺ m/z 415 (100), 417 (60).

### Example 6

### Synthesis of (2-Chloropyrid-3-yl)(6-chloro-1H-indol-3-yl)methanone from 4-Chloro-2-nitrotoluene

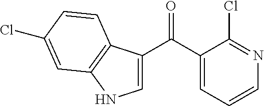 [SMILES: O=C(c1cccnc1Cl)c1c[nH]c2cc(Cl)ccc12]

### 4-Chloro-1-[(2-dimethylamino)ethenyl]-2-nitrobenzene

4-Chloro-2-nitrotoluene (Aldrich, 6.29 g, 36.6 mmol) in dry DMF (21 mL) was treated with neat N,N-dimethylformamide dimethylacetal (Aldrich; 6.5 mL, g, mmol). The reaction was heated at 110° C. overnight and then conc. to dryness. The crude product was triturated with hexanes to give 6.00 g of the product as a dark solid.

### 1-(2-Chloropyrid-3-yl)-2-(4-chloro-2-nitrophenyl)-α-[(dimethylamino)methylene]-ethanone

4-Chloro-1-[(2-dimethylamino)ethenyl]-2-nitrobenzene (1.032 g, 4.55 mmol) in toluene (10 mL) was treated with Et₃N (650 μL, 472 mg, 4.66 mmol). Solid 2-chloronicotinoyl chloride (802 mg, 4.56 mmol) was then added in portions. The resulting deep red solution was stirred at rt and then at 90° C. for 10 min. After cooling to rt, the reaction was diluted with 20 mL of toluene, filtered and the solid collected was washed with toluene (10 mL) and water (10 mL) affording 1.22 g of the product (73% yield). TOF MS ES⁺ 366, 368 (M+H⁺), 388, 390 (M+Na⁺).

Ref: WO 2010/051373 page 72 and WO 2011/137342

### (2-Chloropyrid-3-yl)(6-chloro-1H-indol-3-yl)methanone

1-(2-Chloropyrid-3-yl)-2-(4-chloro-2-nitrophenyl)-α-[(dimethylamino)methylene]ethanone (217 mg, 0.595 mmol) in THF (20 mL) and MeOH (15 mL) was treated with solid NH₄Cl (318 mg, 5.95 mmol) and Zn dust (409 mg, 6.25 mmol). After stirring at rt for 90 min, the mixture was filtered through Celite and the Celite was washed with THF (2×10 mL). The solvent was removed in vacuo and the residue was triturated with CH₂Cl₂ affording 92 mg of the product as a solid. TOF MS ES⁺ m/z 291, 293.

### Oocyte Electrophysiology

The modulation of compounds of the invention was determined in oocytes expressing human α7 nAChRs as described above. Preferred compounds exhibited at least 100% modulation of the nicotine EC₅ at 10 μM. Compounds of paragraph [0060] exhibited at least 100% modulation of the nicotine EC₅ at 10 μM. More preferred compounds exhibited at least 500% modulation of the nicotine EC₅ at 10 μM. Even more preferred compounds exhibited at least 1000% modulation of the nicotine EC₅ at 10 μM.

The patents and publications listed herein describe the general skill in the art and are hereby incorporated by reference in their entireties for all purposes and to the same extent as if each was specifically and individually indicated to be incorporated by reference. In the case of any conflict between a cited reference and this specification, the specification shall control. In describing embodiments of the present application, specific terminology is employed for the sake of clarity. However, the invention is not intended to be limited to the specific terminology so selected. Nothing in this specification should be considered as limiting the scope of the present invention. All examples presented are representative and non-limiting. The above-described embodiments may be modified or varied, without departing from the invention, as appreciated by those skilled in the art in light of the above teachings. It is therefore to be understood that, within the scope of the claims and their equivalents, the invention may be practiced otherwise than as specifically described.

What is claimed is:

1. A compound of Formula I: 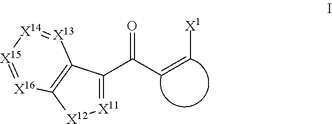
or a pharmaceutically acceptable salt, solvate or prodrug thereof, wherein: 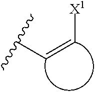
is a heteroaryl group selected from the group consisting of: 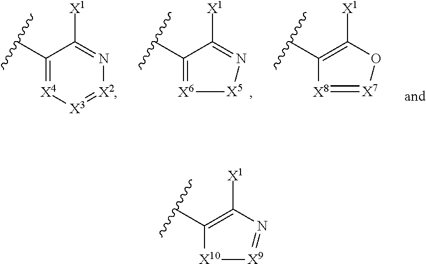
X¹ is O—R¹ or NH—R¹;
X² is N or C—R²;
X³ is N or C—R³;
X⁴ is N or C—R⁴;
X⁵ is O, S or N—R⁵;
X⁶ is N or N—R⁶;
X⁷ is N or C—R⁷;
X⁸ is N or C—R⁸;
X⁹ is N or C—R⁹;
X¹⁰ is O, S or N—R¹⁰;
X¹¹ is N or C—R¹¹;
X¹² is O, S or N—R¹²;
X¹³ is N or C—R¹³;
X¹⁴ is N or C—R¹⁴;
X¹⁵ is N or C—R¹⁵;
X¹⁶ is N or C—R¹⁶;
R¹ is selected from the group consisting of C₁₋₈ alkyl, C₂₋₈ alkenyl, C₂₋₈ alkynyl, and C₁₋₈ haloalkyl, each optionally substituted; or
R¹ is selected from the group consisting of aryl, heteroaryl, arylalkyl, heteroarylalkyl, C₃₋₈ cycloalkyl, cycloalkenyl, heterocycloalkyl and heterocycloalkenyl, each optionally substituted; and
R², R³, R⁴, R⁶, R⁷, R⁸, R⁹, R¹¹, R¹³, R¹⁴, R¹⁵ and R¹⁶ are each independently selected from the group consisting of hydrogen, halogen, nitro, cyano, hydroxyl, amino, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl, heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₈ alkamino, C₁₋₈ haloalkamino, dialkylamino, alkenylamino, alkynylamino, arylamino, heteroarylamino, C₃₋₈ cycloalkamino, cycloalkenylamino, heterocycloalkylamino, heterocycloalkenylamino, C₁₋₈ alkthio, C₁₋₈ haloalkthio, alkenylthio, alkynylthio, arylthio, heteroarylthio, C₃₋₈ cycloalkthio, cycloalkenylthio, heterocycloalkylthio, heterocycloalkenylthio, —C(═O)R¹⁷, —N(R¹⁸)C(═O)R¹⁹, —OC(═O)R¹⁹, —N(R¹⁸)S(═O)₂R¹⁹, —S(═O)₂R¹⁷, and —S(═O)R¹⁷, each optionally substituted; and
R⁵, R¹⁰ and R¹² are independently selected from the group consisting of hydrogen, C₁₋₈ alkyl, C₃₋₈ cycloalkyl, C₃₋₈ alkenyl, C₃₋₈ alkynyl, C₁₋₈ haloalkyl, aryl, and heteroaryl; and
R² and R³, or R³ and R⁴, or R⁵ and R⁶, or R⁷ and R⁸, or R⁹ and R¹⁰, or R¹³ and R¹⁴, or R¹⁴ and R¹⁵ or R¹⁵ and R¹⁶ are taken together with the carbon atoms to which they are attached to form an unsubstituted or substituted fused 5 or 6-membered unsaturated or partially unsaturated ring optionally interrupted by one —O—, —NR²⁰—, —S—, —SO— or —SO₂—; and
each R¹⁷ is independently selected from the group consisting of hydroxyl, amino, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₆ alkylamino, C₁₋₈ haloalkylamino, dialkylamino, alkenylamino, alkynylamino, arylamino, heteroarylamino, C₃₋₈ cycloalkylamino, cycloalkenylamino, heterocycloalkylamino, and heterocycloalkenylamino, each optionally substituted; and
each R¹⁸ is independently selected from the group consisting of hydrogen, hydroxyl, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, and heterocycloalkenyloxy, each optionally substituted; and
each R¹⁹ is independently selected from the group consisting of amino, C₁₋₈ alkyl, C₂₋₆ alkenyl, C₂₋₆ alkynyl, C₁₋₆ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₈ alkylamino, C₁₋₈ haloalkylamino, dialkylamino, alkenylamino, alkynylamino, arylamino, heteroarylamino, C₃₋₈ cycloalkylamino, cycloalkenylamino, heterocycloalkylamino, and heterocycloalkenylamino, each optionally substituted; and
R²⁰ is selected from the group consisting of hydrogen, C₁₋₈ alkyl, C₃₋₈ cycloalkyl, C₃₋₈ alkenyl, C₃₋₈ alkynyl, C₁₋₈ haloalkyl, aryl, and heteroaryl.

2. A compound of Formula II: 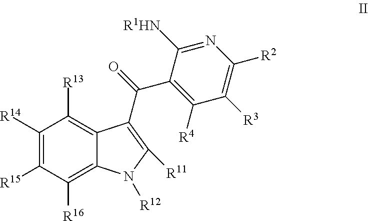
or a pharmaceutically acceptable salt, prodrug or solvate thereof, wherein:
R¹ is selected from the group consisting of C₁₋₈ alkyl, C₂₋₈ alkenyl, C₂₋₈ alkynyl, and C₁₋₈ haloalkyl, each optionally substituted; or
R¹ is selected from the group consisting of aryl, heteroaryl, arylalkyl, heteroarylalkyl, C₃₋₈ cycloalkyl, cycloalkenyl, heterocycloalkyl and heterocycloalkenyl, each optionally substituted; and
R², R³, R⁴, R¹¹, R¹³, R¹⁴, R¹⁵ and R¹⁶ are each independently selected from the group consisting of hydrogen, halogen, nitro, cyano, hydroxyl, amino, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₈ alkamino, C₁₋₈ haloalkamino, dialkylamino, alkenylamino, alkynylamino, arylamino, heteroarylamino, C₃₋₈ cycloalkamino, cycloalkenylamino, heterocycloalkylamino, heterocycloalkenylamino, C₁₋₈ alkthio, C₁₋₈ haloalkthio, alkenylthio, alkynylthio, arylthio, heteroarylthio, C₃₋₈ cycloalkthio, cycloalkenylthio, heterocycloalkylthio, heterocycloalkenylthio, —C(═O)R¹⁷, —N(R¹⁸)C(═O)R¹⁹, —OC(═O)R¹⁹, —N(R¹⁸)S(═O)₂R¹⁹, S(═O)₂R¹⁷, and —S(═O)R¹⁷, each optionally substituted; and
R¹² is selected from the group consisting of hydrogen, C₁₋₈ alkyl, C₃₋₈ cycloalkyl, C₃₋₈ alkenyl, C₃₋₈ alkynyl, C₁₋₈ haloalkyl, aryl, and heteroaryl; and
R² and R³, or R³ and R⁴, or R¹³ and R¹⁴, or R¹⁴ and R¹⁵ or R¹⁵ and R¹⁶ are taken together with the carbon atoms to which they are attached to form an unsubstituted or substituted fused 5 or 6-membered unsaturated or partially unsaturated ring optionally interrupted by one —O—, —NR²O—, —S—, —SO— or —SO₂—; and
each R¹⁷ is independently selected from the group consisting of hydroxyl, amino, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₆ alkylamino, C₁₋₈ haloalkylamino, dialkylamino, alkenylamino, alkynylamino, arylamino, heteroarylamino, C₃₋₈ cycloalkylamino, cycloalkenylamino, heterocycloalkylamino, and heterocycloalkenylamino, each optionally substituted; and
each R¹⁸ is independently selected from the group consisting of hydrogen, hydroxyl, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, and heterocycloalkenyloxy, each optionally substituted; and
each R¹⁹ is independently selected from the group consisting of amino, C₁₋₈ alkyl, C₂₋₆ alkenyl, C₂₋₆ alkynyl, C₁₋₆ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₈ alkylamino, C₁₋₈ haloalkylamino, dialkylamino, alkenylamino, alkynylamino, arylamino, heteroarylamino, C₃₋₈ cycloalkylamino, cycloalkenylamino, heterocycloalkylamino, and heterocycloalkenylamino, each optionally substituted; and
R²⁰ is selected from the group consisting of hydrogen, C₁₋₈ alkyl, C₃₋₈ cycloalkyl, C₃₋₈ alkenyl, C₃₋₈ alkynyl, C₁₋₈ haloalkyl, aryl, and heteroaryl.

3. A compound of Formula III: 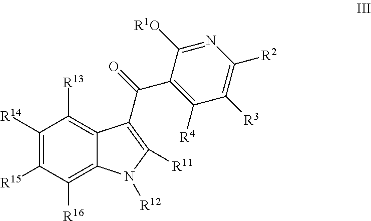
or a pharmaceutically acceptable salt, prodrug or solvate thereof, wherein:
R¹ is selected from the group consisting of C₁₋₈ alkyl, C₂₋₈ alkenyl, C₂₋₈ alkynyl, and C₁₋₈ haloalkyl, each optionally substituted; or
R¹ is selected from the group consisting of aryl, heteroaryl, arylalkyl, heteroarylalkyl, C₃₋₈ cycloalkyl, cycloalkenyl, heterocycloalkyl and heterocycloalkenyl, each optionally substituted; and
R², R³, R⁴, R¹¹, R¹³, R¹⁴, R¹⁵ and R¹⁶ are each independently selected from the group consisting of hydrogen, halogen, nitro, cyano, hydroxyl, amino, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₈ alkamino, C₁₋₈ haloalkamino, dialkylamino, alkenylamino, alkynylamino, arylamino, heteroarylamino, C₃₋₈ cycloalkamino, cycloalkenylamino, heterocycloalkylamino, heterocycloalkenylamino, C₁₋₈ alkthio, C₁₋₈ haloalkthio, alkenylthio, alkynylthio, arylthio, heteroarylthio, C₃₋₈ cycloalkthio, cycloalkenylthio, heterocycloalkylthio, heterocycloalkenylthio, —C(═O)R¹⁷, —N(R¹⁸)C(═O)R¹⁹, —OC(═O)R¹⁹, —N(R¹⁸)S(═O)₂R¹⁹, S(═O)₂R¹⁷, and —S(═O)R¹⁷, each optionally substituted; and
R¹² is selected from the group consisting of hydrogen, C₁₋₈ alkyl, C₃₋₈ cycloalkyl, C₃₋₈ alkenyl, C₃₋₈ alkynyl, C₁₋₈ haloalkyl, aryl, and heteroaryl; and
R² and R³, or R³ and R⁴, or R¹³ and R¹⁴, or R¹⁴ and R¹⁵ or R¹⁵ and R¹⁶ are taken together with the carbon atoms to which they are attached to form an unsubstituted or substituted fused 5 or 6-membered unsaturated or partially unsaturated ring optionally interrupted by one —O—, —NR²O—, —S—, —SO— or —SO₂—; and
each R¹⁷ is independently selected from the group consisting of hydroxyl, amino, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₆ alkylamino, C₁₋₈ haloalkylamino, dialkylamino, alkenylamino, alkynylamino, arylamino, heteroarylamino, C₃₋₈ cycloalkylamino, cycloalkenylamino, heterocycloalkylamino, and heterocycloalkenylamino, each optionally substituted; and
each R¹⁸ is independently selected from the group consisting of hydrogen, hydroxyl, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, and heterocycloalkenyloxy, each optionally substituted; and
each R¹⁹ is independently selected from the group consisting of amino, C₁₋₈ alkyl, C₂₋₆ alkenyl, C₂₋₆ alkynyl, C₁₋₆ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₈ alkylamino, C₁₋₈ haloalkylamino, dialkylamino, alkenylamino, alkynylamino, arylamino, heteroarylamino, C₃₋₈ cycloalkylamino, cycloalkenylamino, heterocycloalkylamino, and heterocycloalkenylamino, each optionally substituted; and
R²⁰ is selected from the group consisting of hydrogen, C₁₋₈ alkyl, C₃₋₈ cycloalkyl, C₃₋₈ alkenyl, C₃₋₈ alkynyl, C₁₋₈ haloalkyl, aryl, and heteroaryl.

4. A compound of Formula IV: 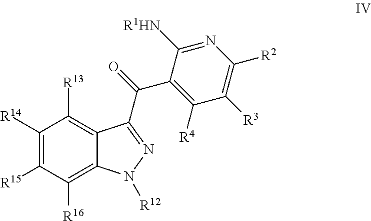
or a pharmaceutically acceptable salt, prodrug or solvate thereof, wherein:
R¹ is selected from the group consisting of C₁₋₈ alkyl, C₂₋₈ alkenyl, C₂₋₈ alkynyl, and C₁₋₈ haloalkyl, each optionally substituted; or
R¹ is selected from the group consisting of aryl, heteroaryl, arylalkyl, heteroarylalkyl, C₃₋₈ cycloalkyl, cycloalkenyl, heterocycloalkyl and heterocycloalkenyl, each optionally substituted; and
R², R³, R⁴, R¹³, R¹⁴, R¹⁵ and R¹⁶ are each independently selected from the group consisting of hydrogen, halogen, nitro, cyano, hydroxyl, amino, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₈ alkamino, C₁₋₈ haloalkamino, dialkylamino, alkenylamino, alkynylamino, arylamino, heteroarylamino, C₃₋₈ cycloalkamino, cycloalkenylamino, heterocycloalkylamino, heterocycloalkenylamino, C₁₋₈ alkthio, C₁₋₈ haloalkthio, alkenylthio, alkynylthio, arylthio, heteroarylthio, C₃₋₈ cycloalkthio, cycloalkenylthio, heterocycloalkylthio, heterocycloalkenylthio, —C(═O)R¹⁷, —N(R¹⁸)C(═O)R¹⁹, —OC(═O)R¹⁹, —N(R¹⁸)S(═O)₂R¹⁹, —S(═O)₂R¹⁷, and —S(═O)R¹⁷, each optionally substituted; and
R¹² is selected from the group consisting of hydrogen, C₁₋₈ alkyl, C₃₋₈ cycloalkyl, C₃₋₈ alkenyl, C₃₋₈ alkynyl, C₁₋₈ haloalkyl, aryl, and heteroaryl; and
R² and R³, or R³ and R⁴, or R¹³ and R¹⁴, or R¹⁴ and R¹⁵ or R¹⁵ and R¹⁶ are taken together with the carbon atoms to which they are attached to form an unsubstituted or substituted fused 5 or 6-membered unsaturated or partially unsaturated ring optionally interrupted by one —O—, —NR²O—, —S—, —SO— or —SO₂—; and
each R¹⁷ is independently selected from the group consisting of hydroxyl, amino, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₆ alkylamino, C₁₋₈ haloalkylamino, dialkylamino, alkenylamino, alkynylamino, arylamino, heteroarylamino, C₃₋₈ cycloalkylamino, cycloalkenylamino, heterocycloalkylamino, and heterocycloalkenylamino, each optionally substituted; and
each R¹⁸ is independently selected from the group consisting of hydrogen, hydroxyl, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, and heterocycloalkenyloxy, each optionally substituted; and
each R¹⁹ is independently selected from the group consisting of amino, C₁₋₈ alkyl, C₂₋₆ alkenyl, C₂₋₆ alkynyl, C₁₋₆ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₈ alkylamino, C₁₋₈ haloalkylamino, dialkylamino, alkenylamino, alkynylamino, arylamino, heteroarylamino, C₃₋₈ cycloalkylamino, cycloalkenylamino, heterocycloalkylamino, and heterocycloalkenylamino, each optionally substituted; and
R²⁰ is selected from the group consisting of hydrogen, C₁₋₈ alkyl, C₃₋₈ cycloalkyl, C₃₋₈ alkenyl, C₃₋₈ alkynyl, C₁₋₈ haloalkyl, aryl, and heteroaryl.

5. A compound of Formula V: 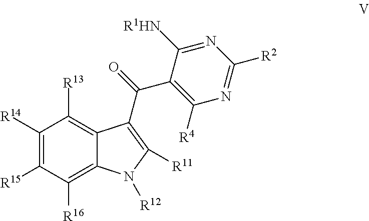
or a pharmaceutically acceptable salt, prodrug or solvate thereof, wherein:
R¹ is selected from the group consisting of C₁₋₈ alkyl, C₂₋₈ alkenyl, C₂₋₈ alkynyl, and C₁₋₈ haloalkyl, each optionally substituted; or
R¹ is selected from the group consisting of aryl, heteroaryl, arylalkyl, heteroarylalkyl, C₃₋₈ cycloalkyl, cycloalkenyl, heterocycloalkyl and heterocycloalkenyl, each optionally substituted; and
R², R⁴, R¹¹, R¹³, R¹⁴, R¹⁵ and R¹⁶ are each independently selected from the group consisting of hydrogen, halogen, nitro, cyano, hydroxyl, amino, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₈ alkamino, C₁₋₈ haloalkamino, dialkylamino, alkenylamino, alkynylamino, arylamino, heteroarylamino, C₃₋₈ cycloalkamino, cycloalkenylamino, heterocycloalkylamino, heterocycloalkenylamino, C₁₋₈ alkthio, C₁₋₈ haloalkthio, alkenylthio, alkynylthio, arylthio, heteroarylthio, C₃₋₈ cycloalkthio, cycloalkenylthio, heterocycloalkylthio, heterocycloalkenylthio, —C(═O)R¹⁷, —N(R¹⁸)C(═O)R¹⁹, —OC(═O)R¹⁹, —N(R¹⁸)S(═O)₂R¹⁹, S(═O)₂R¹⁷, and —S(═O)R¹⁷, each optionally substituted; and
R¹² is selected from the group consisting of hydrogen, C₁₋₈ alkyl, C₃₋₈ cycloalkyl, C₃₋₈ alkenyl, C₃₋₈ alkynyl, C₁₋₈ haloalkyl, aryl, and heteroaryl; and
R¹³ and R¹⁴, or R¹⁴ and R¹⁵ or R¹⁵ and R¹⁶ are taken together with the carbon atoms to which they are attached to form an unsubstituted or substituted fused 5 or 6-membered unsaturated or partially unsaturated ring optionally interrupted by one —O—, —NR²O—, —S—, —SO— or —SO₂—; and
each R¹⁷ is independently selected from the group consisting of hydroxyl, amino, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₆ alkylamino, C₁₋₈ haloalkylamino, dialkylamino, alkenylamino, alkynylamino, arylamino, heteroarylamino, C₃₋₈ cycloalkylamino, cycloalkenylamino, heterocycloalkylamino, and heterocycloalkenylamino, each optionally substituted; and
each R¹⁸ is independently selected from the group consisting of hydrogen, hydroxyl, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, and heterocycloalkenyloxy, each optionally substituted; and
each R¹⁹ is independently selected from the group consisting of amino, C₁₋₈ alkyl, C₂₋₆ alkenyl, C₂₋₆ alkynyl, C₁₋₆ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₈ alkylamino, C₁₋₈ haloalkylamino, dialkylamino, alkenylamino, alkynylamino, arylamino, heteroarylamino, C₃₋₈ cycloalkylamino, cycloalkenylamino, heterocycloalkylamino, and heterocycloalkenylamino, each optionally substituted; and
R²⁰ is selected from the group consisting of hydrogen, C₁₋₈ alkyl, C₃₋₈ cycloalkyl, C₃₋₈ alkenyl, C₃₋₈ alkynyl, C₁₋₈ haloalkyl, aryl, and heteroaryl.

6. A compound of Formula VI: 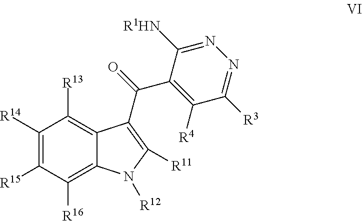
or a pharmaceutically acceptable salt, prodrug or solvate thereof, wherein:
R¹ is selected from the group consisting of C₁₋₈ alkyl, C₂₋₈ alkenyl, C₂₋₈ alkynyl, and C₁₋₈ haloalkyl, each optionally substituted; or
R¹ is selected from the group consisting of aryl, heteroaryl, arylalkyl, heteroarylalkyl, C₃₋₈ cycloalkyl, cycloalkenyl, heterocycloalkyl and heterocycloalkenyl, each optionally substituted; and
R³, R⁴, R¹¹, R¹³, R¹⁴, R¹⁵ and R¹⁶ are each independently selected from the group consisting of hydrogen, halogen, nitro, cyano, hydroxyl, amino, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₈ alkamino, C₁₋₈ haloalkamino, dialkylamino, alkenylamino, alkynylamino, arylamino, heteroarylamino, C₃₋₈ cycloalkamino, cycloalkenylamino, heterocycloalkylamino, heterocycloalkenylamino, C₁₋₈ alkthio, C₁₋₈ haloalkthio, alkenylthio, alkynylthio, arylthio, heteroarylthio, C₃₋₈ cycloalkthio, cycloalkenylthio, heterocycloalkylthio, heterocycloalkenylthio, —C(═O)R¹⁷, —N(R¹⁸)C(═O)R¹⁹, —OC(═O)R¹⁹, —N(R¹⁸)S(═O)₂R¹⁹, S(═O)₂R¹⁷, and —S(═O)R¹⁷, each optionally substituted; and
R¹² is selected from the group consisting of hydrogen, C₁₋₈ alkyl, C₃₋₈ cycloalkyl, C₃₋₈ alkenyl, C₃₋₈ alkynyl, C₁₋₈ haloalkyl, aryl, and heteroaryl; and
R³ and R⁴, or R¹³ and R¹⁴, or R¹⁴ and R¹⁵ or R¹⁵ and R¹⁶ are taken together with the carbon atoms to which they are attached to form an unsubstituted or substituted fused 5 or 6-membered unsaturated or partially unsaturated ring optionally interrupted by one —O—, —NR²O—, —S—, —SO— or —SO₂—; and
each R¹⁷ is independently selected from the group consisting of hydroxyl, amino, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₆ alkylamino, C₁₋₈ haloalkylamino, dialkylamino, alkenylamino, alkynylamino, arylamino, heteroarylamino, C₃₋₈ cycloalkylamino, cycloalkenylamino, heterocycloalkylamino, and heterocycloalkenylamino, each optionally substituted; and
each R¹⁸ is independently selected from the group consisting of hydrogen, hydroxyl, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, and heterocycloalkenyloxy, each optionally substituted; and
each R¹⁹ is independently selected from the group consisting of amino, C₁₋₈ alkyl, C₂₋₆ alkenyl, C₂₋₆ alkynyl, C₁₋₆ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₈ alkylamino, C₁₋₈ haloalkylamino, dialkylamino, alkenylamino, alkynylamino, arylamino, heteroarylamino, C₃₋₈ cycloalkylamino, cycloalkenylamino, heterocycloalkylamino, and heterocycloalkenylamino, each optionally substituted; and
R²⁰ is selected from the group consisting of hydrogen, C₁₋₈ alkyl, C₃₋₈ cycloalkyl, C₃₋₈ alkenyl, C₃₋₈ alkynyl, C₁₋₈ haloalkyl, aryl, and heteroaryl.

7. A compound of Formula VII: 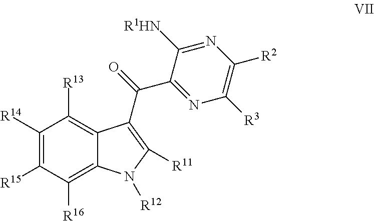
or a pharmaceutically acceptable salt, prodrug or solvate thereof, wherein:
R¹ is selected from the group consisting of C₁₋₈ alkyl, C₂₋₈ alkenyl, C₂₋₈ alkynyl, and C₁₋₈ haloalkyl, each optionally substituted; or
R¹ is selected from the group consisting of aryl, heteroaryl, arylalkyl, heteroarylalkyl, C₃₋₈ cycloalkyl, cycloalkenyl, heterocycloalkyl and heterocycloalkenyl, each optionally substituted; and
R², R³, R¹¹, R¹³, R¹⁴, R¹⁵ and R¹⁶ are each independently selected from the group consisting of hydrogen, halogen, nitro, cyano, hydroxyl, amino, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₈ alkamino, C₁₋₈ haloalkamino, dialkylamino, alkenylamino, alkynylamino, arylamino, heteroarylamino, C₃₋₈ cycloalkamino, cycloalkenylamino, heterocycloalkylamino, heterocycloalkenylamino, C₁₋₈ alkthio, C₁₋₈ haloalkthio, alkenylthio, alkynylthio, arylthio, heteroarylthio, C₃₋₈ cycloalkthio, cycloalkenylthio, heterocycloalkylthio, heterocycloalkenylthio, —C(═O)R¹⁷, —N(R¹⁸)C(═O)R¹⁹, —OC(═O)R¹⁹, —N(R¹⁸)S(═O)₂R¹⁹, S(═O)₂R¹⁷, and —S(═O)R¹⁷, each optionally substituted; and
R¹² is selected from the group consisting of hydrogen, C₁₋₈ alkyl, C₃₋₈ cycloalkyl, C₃₋₈ alkenyl, C₃₋₈ alkynyl, C₁₋₈ haloalkyl, aryl, and heteroaryl; and
R² and R³, or R¹³ and R¹⁴, or R¹⁴ and R¹⁵ or R¹⁵ and R¹⁶ are taken together with the carbon atoms to which they are attached to form an unsubstituted or substituted fused 5 or 6-membered unsaturated or partially unsaturated ring optionally interrupted by one —O—, —NR²O—, —S—, —SO— or —SO₂—; and
each R¹⁷ is independently selected from the group consisting of hydroxyl, amino, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₆ alkylamino, C₁₋₈ haloalkylamino, dialkylamino, alkenylamino, alkynylamino, arylamino, heteroarylamino, C₃₋₈ cycloalkylamino, cycloalkenylamino, heterocycloalkylamino, and heterocycloalkenylamino, each optionally substituted; and
each R¹⁸ is independently selected from the group consisting of hydrogen, hydroxyl, C₁₋₈ alkyl, C₂₋₈ alkenyl, C₂₋₈ alkynyl, C₁₋₈ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, and heterocycloalkenyloxy, each optionally substituted; and
each R¹⁹ is independently selected from the group consisting of amino, C₁₋₈ alkyl, C₂₋₆ alkenyl, C₂₋₆ alkynyl, C₁₋₆ haloalkyl, aryl, heteroaryl, C₃₋₈ cycloalkyl, cycloalkenyl, heterocycloalkyl heterocycloalkenyl, C₁₋₈ alkoxy, C₁₋₈ haloalkoxy, alkenyloxy, alkynyloxy, aryloxy, heteroaryloxy, C₃₋₈ cycloalkoxy, cycloalkenyloxy, heterocycloalkyloxy, heterocycloalkenyloxy, C₁₋₈ alkylamino, C₁₋₈ haloalkylamino, dialkylamino, alkenylamino, alkynylamino, arylamino, heteroarylamino, C₃₋₈ cycloalkylamino, cycloalkenylamino, heterocycloalkylamino, and heterocycloalkenylamino, each optionally substituted; and
R²⁰ is selected from the group consisting of hydrogen, C₁₋₈ alkyl, C₃₋₈ cycloalkyl, C₃₋₈ alkenyl, C₃₋₈ alkynyl, C₁₋₈ haloalkyl, aryl, and heteroaryl.

8. The compound according to any one of claims 2, 4-7 wherein:
R¹ is selected from the group consisting of C₁₋₈ alkyl and C₁₋₈ haloalkyl, each optionally substituted; or
R¹ is selected from the group consisting of aryl, heteroaryl, arylalkyl, heteroarylalkyl, C₃₋₈ cycloalkyl, cycloalkenyl, heterocycloalkyl and heterocycloalkenyl, each optionally substituted; and
R², R³, R⁴, R¹¹, R¹³, R¹⁴, R¹⁵ and R¹⁶ are each independently selected from the group consisting of hydrogen, halogen, cyano, amino, C₁₋₈ alkyl, C₁₋₈ haloalkyl, C₃₋₈ cycloalkyl, cycloalkenyl, heterocycloalkyl heterocycloalkenyl, C₁₋₈ alkoxy, C₁₋₈ haloalkoxy, C₃₋₈ cycloalkoxy, C₁₋₈ alkamino, dialkylamino, C₃₋₈ cycloalkamino, cycloalkenylamino, heterocycloalkylamino, heterocycloalkenylamino, C₁₋₈ alkthio, C₁₋₈ haloalkthio, and C₃₋₈ cycloalkthio; and
R¹² is selected from the group consisting of hydrogen, C₁₋₈ alkyl, C₃₋₈ cycloalkyl, and C₁₋₈ haloalkyl,
and pharmaceutically acceptable salts and prodrugs thereof.

9. The compound of claim 8 wherein:
R¹ is selected from the group consisting of arylalkyl and heteroarylalkyl, each optionally substituted;
R¹² is selected from the group consisting of hydrogen and C₁₋₈ alkyl;
R¹³ is hydrogen; and
R¹⁴, R¹⁵ and R¹⁶ are each independently selected from the group consisting of hydrogen, halogen, C₁₋₈ alkyl, and C₁₋₈ haloalkyl; and pharmaceutically acceptable salts, solvates, and prodrugs thereof.

10. The compound of claim 9 wherein:
R is an optionally substituted arylalkyl;
R¹² is selected from the group consisting of hydrogen and C₁₋₈ alkyl;
R¹³ is hydrogen;
R¹⁴ and R¹⁵ are each independently selected from the group consisting of hydrogen and halogen; and
R¹⁶ is hydrogen; and pharmaceutically acceptable salts, solvates, and prodrugs thereof.

11. The compound according to any one of claims 8-10 wherein:
R is an optionally substituted benzyl; and pharmaceutically acceptable salts, solvates, and prodrugs thereof.

12. A compound of claim 1 selected from:
(6-chloro-1H-indol-3-yl)[2-(cyclopentylamino)pyridine-3-yl]methanone;
[2-(benzylamino)pyridine-3-yl](6-chloro-1H-indol-3-yl)methanone;
(6-chloro-1H-indol-3-yl)[2-(pyridine-2-ylmethylamino)pyridine-3-yl]methanone;
(6-chloro-1H-indol-3-yl)[2-(phenethylamino)pyridine-3-yl]methanone;
[2-(benzylamino)pyridine-3-yl](6-fluoro-1H-indol-3-yl)methanone;
(6-chloro-1H-indol-3-yl)[2-(pyridine-4-ylmethylamino)pyridine-3-yl]methanone;
[2-benzylamino)pyridine-3-yl](5-chloro-1H-indol-3-yl)methanone;
(5-chloro-1H-indol-3-yl)[2-(phenethylamino)pyridine-3-yl]methanone;
(5-chloro-1H-indol-3-yl)[2-(phenylamino)pyridine-3-yl]methanone;
(6-chloro-1H-indol-3-yl)[2-[(4-fluorophenyl)amino]pyridine-3-yl]]methanone;
(6-chloro-1H-indol-3-yl)[(2-phenylamino)pyridine-3-yl]methanone;
[2-(benzylamino)pyridine-3-yl](7-chloro-1H-indol-3-yl)methanone;
(6-chloro-1H-indol-3-yl)[2-(4-fluorobenzylamino)pyridine-3-yl]methanone;
(6-chloro-1H-indol-3-yl)[2-(4-methoxybenzylamino)pyridine-3-yl]methanone;
(6-chloro-1H-indol-3-yl)[2-(3,4-difluorobenzylamino)pyridine-3-yl]methanone;
(6-chloro-1H-indol-3-yl)[2-(2,4-difluorobenzylamino)pyridine-3-yl]methanone;
(6-chloro-1H-indol-3-yl)[2-(4-chlorobenzylamino)pyridine-3-yl]methanone;
(6-chloro-1H-indol-3-yl)[2-(4-methylbenzylamino)pyridine-3-yl]methanone;
(6-chloro-1H-indol-3-yl)[2-(cyclohexylmethylamino)pyridine-3-yl]methanone;
(6-chloro-1H-indol-3-yl)[2-(cyclopropylmethylamino)pyridine-3-yl]methanone;
(6-chloro-1H-indol-3-yl)[2-(propylamino)pyridine-3-yl]methanone;
[2-(benzylamino)pyridine-3-yl](6-chloro-1H-indazol-3-yl)methanone;
[2-(benzylamino)pyridine-3-yl](1H-indazol-3-yl)methanone;
[2-(benzylamino)pyridine-3-yl](6-chloro-1-methyl-1H-indol-3-yl)methanone;
[2-(benzylamino)-6-methylpyridine-3-yl](6-chloro-1H-indol-3-yl)methanone;
[2-(tetrahydro-2H-pyran-4-ylamino)pyridine-3-yl](6-chloro-1H-indol-3-)-methanone;
(6-chloro-1H-indol-3-yl)[2-[(4-fluorobenzyl)amino]pyrazin-3-yl]methanone;
and (6-chloro-1H-indol-3-yl)[6-chloro-[3-(4-fluorobenzyl)amino]pyridazin-4-yl]methanone;
and pharmaceutically acceptable salts and prodrugs thereof.

13. A pharmaceutical composition comprising a compound according to any one of claims 1-12, or a pharmaceutically acceptable salt or prodrug thereof, and a pharmaceutically acceptable carrier or diluent.

14. A method for treating a disorder amenable to modulation of α7 nAChR comprising administering to a patient in need of such treatment a compound according to any one of claims 1-12, a pharmaceutically acceptable salt or prodrug thereof or a pharmaceutical composition of claim 13.

15. A method of treating a disorder selected from depression, neurodegenerative diseases, senile dementias, schizophrenia, Alzheimer's disease, learning, cognition and attention deficits, memory loss, Lewy Body dementia, attention-deficit disorder, attention deficit hyperactivity disorder, anxiety, mania, manic depression, Parkinson's disease, Huntington's disease, amyotrophic lateral sclerosis, brain inflammation, cognitive deficit due to traumatic brain injury, autism spectrum disorder, and Tourette's syndrome, comprising administering to a patient in need thereof a compound according to any one of claims 1-12, or a pharmaceutically acceptable salt or prodrug thereof.

16. A method for treating a cognitive disorder related to learning or memory comprising administering to a patient in need of such treatment a compound according to any one of claims 1-12, or a pharmaceutically acceptable salt or prodrug thereof.

17. A method for the treatment of disorders which comprises administering to a patient in need of such treatment a compound according to any one of claims 1-12 or a pharmaceutically acceptable salt or prodrug thereof, with activity for positive allosteric modulation of currents at α7 nAChR receptors in which modulated currents retain the rapid native kinetics and native desensitization of the receptor observed in the absence of said compound, or pharmaceutically acceptable salt or prodrug thereof.

18. The method of claim 15, wherein the disorder is a neurodegenerative disorder.

19. The method of claim 15, wherein the disorder is a senile dementia.

20. The method of claim 15, wherein the disorder is Alzheimer's disease.

21. The method of claim 15, wherein the disorder is schizophrenia.

22. The method of claim 15, wherein the disorder is a mild cognitive impairment.

23. The method of claim 15, wherein the disorder is Parkinson's disease.

24. The method of claim 14, wherein the disorder is inflammation.

25. The method of claim 14, wherein the disorder is an immune system disorder.

26. The method of claim 14, wherein the composition is administered to treat pain, inflammation, septic shock, ulcerative colitis, Crohn's disease, or irritable bowel syndrome.

27. The method of claim 15, wherein the condition treated is autism spectrum disorder.
